(19) United States
(12) Patent Application Publication
(10) Pub. No.: US 2025/0375281 A1
(43) Pub. Date: Dec. 11, 2025

(54) MESH IMPLANT DEVICE, SYSTEM, KIT AND METHODS OF USE

(71) Applicant: Sun Parasol LLC, Dobbs Ferry, NY (US)

(72) Inventors: Sundaram Ravikumar, Briarcliff Manor, NY (US); Guy Osborne, Trumbull, CT (US)

(73) Assignee: Sun Scientific Inc., Dobbs Ferry, NY (US)

(21) Appl. No.: 18/877,093

(22) PCT Filed: Jun. 23, 2023
(86) PCT No.: PCT/US2023/026121

§ 371 Date: Dec. 19, 2024

Related U.S. Application Data
(60) Provisional application No. 63/355,066, filed on Jun. 23, 2022.
(60) Provisional application No. 63/359,445, filed on Jul. 8, 2022.

(51) Int. Cl. A61F 2/00; A61B 17/04

(52) U.S. Cl. CPC A61F 2/0063; A61B 17/0469; A61F 2002/0068; A61F 2002/0072; A61F 2220/0008; A61F 2220/0016; A61F 2230/0093

(57) ABSTRACT

A mesh deployment device includes a hollow housing within which mesh connected to a series of supports is housed and then activated during deployment, which supports are removed from the mesh once the mesh is located on the target hernia area and the supports are retracted back into the hollow housing and out of the body cavity. The mesh deployment device may include an optional port closure suture system on the mesh, and may also optionally include a series of attachment means on the mesh to connected to the target body cavity location.

### RELATED APPLICATIONS

The present application claims priority from PCT application Serial No. PCT/US23/26121 entitled ‘Mesh Implant Device, System, Kit and Methods of Use” filed Jun. 17, 2023 claiming priority from provisional application Ser. No. 63/355,066 filed Jun. 23, 2022 and entitled “Port Closure Device and Methods of Use”, and provisional application Ser. No. 63/359,445 filed Jul. 8, 2022 and entitled “Mesh Implant Device, Kit and Methods of Use”, the entire contents of both which are hereby incorporated herein by reference for all purposes.

### FIELD OF THE INVENTION

The present invention relates to a mesh implant device, a system, a kit containing the device, and method of use of the device.

### BACKGROUND OF THE INVENTION

The present invention relates to a mesh implant device. The mesh implant device may also be used to close ports or incisions points within the body during or at the conclusion of a surgical procedures, such as the umbilicus, if it includes the optional suture attached to the mesh implant. The mesh implant device includes a mesh implant and the inventive device may be used in the reconstruction of soft tissue defects, such as a hernia. Such mesh implants are in particular used in the repair of defects in the abdominal wall, which may be a result from trauma, tumor resection, prolapse or hernia. Other uses are known for a mesh implant and may be employed with the inventive device.

Within the field of surgical repair of soft tissue defects, use is often made of a mesh implant made of a non-resorbable material that is inserted to cover the area of the tissue defect. The mesh implant is used in order to support the regenerating tissue (e.g., hernia defect) and works by mechanical closure of the defect and by inducing a strong scar fibrous tissue around the mesh implant. Such a mesh implant is most often made of various plastics, which are known to stay biostable and safe for a number of years after implantation. However, such mesh implants may crumple up and loose its tissue supporting function over time.

One such use of a mesh implant is during a hernia surgical procedure. A hernia is an abnormal protrusion of a peritoneal-lined sac through the musculoaponeurotic covering of the abdomen, the most common site for a hernia being the groin though very often the hernia is located in the abdominal area. A hernia is a weakness or tear in the abdominal muscles which allows fatty tissue or an organ such as the intestines to protrude through the weak area. This can cause a noticeable bulge under the skin, and the pressure of tissue pushing its way through the weakened area can be the source of significant pain and discomfort for the patient. Symptoms can feel worse when the individual stands for long periods, during urination or a bowel movement, or when lifting heavy objects. Types of hernia include, without limitation, inguinal hernia or a femoral hernia, hiatal hernia, umbilical hernia and incisional hernia, the latter being a hernia that pushes through a past surgical incision or operation.

More than one million hernia operations were performed in the United States, according to the U.S. Food and Drug Administration (FDA) rising to almost 1.5 million, and worldwide these surgeries are estimated to top 20 million annually, with the numbers rising each year. Notably, the recurrence rate for groin hernias is 1 to 3 percent, while it's 5 to 10 percent for abdominal (ventral) hernias, and 10 to 15 percent for stoma hernias. For the most complex hernias, the recurrence rate is 10 to 20 percent, depending on the nature of the hernia and other factors. One suggested theory in the field is that some patients, due to collagen metabolic disorders, have a genetic predisposition for developing recurrent hernias. Recurrent hernias are a common problem in hernia surgery. Even the best reports indicate from 1% to 4% recurrent hernias after primary surgery, and some authors report figures up to 20%. These figures are much lower when a non-absorbable mesh is utilized in the method of surgery.

Older types of surgical repair of a hernia were through traditional tension repair wherein the surgeon made an incision in the abdomen over the hernia site, pushed any protruding tissue back into correct position within the abdominal cavity, and then stitched the hernia closed. A tension repair is used currently in some instances for children or if the hernia is extremely small in an adult patient. This technique however has several disadvantages. First, the level of discomfort following a tension repair is greater, and the recovery period is longer (about 4-6 weeks) than with a tension-free repair. In addition, there is an approximately 10-15 percent chance that the hernia will recur or happen again.

For cases dealing with large or recurrent hernias the surgeon may employ surgical repair or herniorrhaphy making use of an inert, non-resorbable mesh implant as described above and herein. The mesh implant is inserted within the body cavity through a trocar, typically at the umbilicus or other incision port or point, and various surgical instruments are used (within the same large port or through additional incision points or ports within the fascia and body cavity) to place the mesh implant over and covering the area of the abdominal wall defect without sewing together the surrounding muscles of the target tissue location. This can be done under local or general anesthesia using a laparoscope or an open incision technique. The mesh implant is typically, either sutured into the abdominal wall or fastening using a series of surgical staples or tacks. Many problems occur in such surgical procedures including the space needed to facilitate placement of the mesh implant, use of numerous sutures and staples, as well as the need to close the incision port or area.

Among the laparoscopic techniques used in such conventional surgical procedures are the trans-abdominal pre-peritoneal (TAPP) technique and the totally extra-peritoneal (TEP) technique. With the TAPP technique, the pre-peritoneal space is accessed from the abdominal cavity, whereupon the mesh implant is placed between the peritoneum and the transversalis fascia. With the TEP technique, the mesh implant is again placed in the retroperitoneal space, but the space is accessed without violating the abdominal cavity. An open and minimal invasive technique is the Uchtenstein hernia repair technique, in which the upper edge of the mesh implant is attached to the outer side of the internal oblique and the lower edge of the mesh implant is attached to the aponeurotic tissue covering the pubis.

Another open minimal invasive technique is the mesh-plug technique comprising attaching a mesh implant, as described above in reference to the Lichtenstein technique, but also inserting a plug pushing the peritoneum in a direction towards the abdominal cavity. In an open peritoneal technique, a small 1.5-2 inch incision is made near the hernia bulge. The hernia is then identified and pushed back into the abdomen. The space between the abdominal wall and the peritoneum is freed up. A mesh implant is then placed into this opening so that it covers the hernia, as well as other potential hernia sites. The mesh implant is then sutured into the site and in a separate step the fascia of the patient is then closed with sutures and glue. In an alternate surgical procedure of laparoscopy the surgeon inserts a laparoscope, a thin instrument consisting of a lighted tube with magnifying lenses, through an incision in the abdomen, typically at the umbilicus and can be between about 5 mm to about 30 mm in diameter. Carbon dioxide gas is placed into the abdominal cavity to create a working space. The laparoscope enables the surgeon to examine the hernia and accurately place a mesh plant on the inside of the abdominal wall, not outside as with traditional mesh repair. Through two other small incisions or ports in other locations on the fascia, special operating instruments are used to open up the space between the peritoneum and the abdominal wall, large enough to permit a mesh implant to be placed to cover the hernia hole and the other potential weak areas. The peritoneum is sewn back together again with sutures to keep the mesh implant in place, or surgical staples or tacks may be used to connect the mesh implant. The carbon dioxide gas is removed and the multiple incision points or ports in the abdomen and fascia are closed carefully so that they do also not become hernias over time.

Lastly, a plug and patch repair procedures starts with an incision point or port made over the inguinal hernia. Once the herniated tissue has been pushed back in, then a circular or other shaped mesh implant ping is placed into the hole and the edges of mesh are sutured to the edges of the hole or connected via staples, tacks or the like. Then a second additional flat piece of mesh is placed between two layers of the abdominal wall to provide a more broad-based reinforcement of the hernia repair. This surgical procedure requires two layers of mesh implants over the target area where the hernia is located and increases surgical time and possibility of complications at the surgical site. Current complications from the various known surgical procedures include recurrence as discussed above and other side effects and complications such as injury to the bowl during surgery, swelling and pain in the scrotum or groin after surgery, development of a painful testicle, an infection of the mesh implant requiring removal in a second surgical procedure, and in general a prolonged recovery.

The mesh implant, also referred to as a patch or plug, inserted with any of the above described techniques, is used in order to support the regenerating tissue with minimal tension. It works by mechanical closure of the defect in the abdominal wall and by inducing a strong scar tissue around the mesh implant fibers. The commercially available hernia mesh implants are often made of various, inert, non-resorbable polymeric materials, typically polypropylene, and suffers from the same disadvantages, as described above in connection with mesh implants used for reconstruction of soft tissue defects In general. However, implantation of large pieces of mesh implants in the abdominal wall cavity also leads to considerable restriction thereof. Further, the non-physiological stretching capability of the mesh implants contrast with the highly elastic abdominal wall and can give rise to shearing forces.

Further problems have developed with conventional mesh implants and the suturing or stapling of such mesh implants to the abdominal wall, such as the common plastic “memory recoil ring” bordering the mesh implant may fracture, causing bowel perforations or abnormal connections between the intestines and other organs known as fistulae. This damage not only causes severe pain but can also lead to additional medical complications of infection and even death. In addition, while the mesh implants are typically made of various plastics which are known to stay biostable and safe at least for the usual follow-up time of 5 to IO years after implantation, the possibility of degradation, cracking and breakage within the body remains and certain mesh implants have been the subject of recalls in the United States. On the other hand, permanent surgical implants (metals, silicone, etc.) have been shown to cause side effects in many patients because of corrosion, wearing, migration, chronic inflammation and risk of infection. When the foreign material is placed near sensitive organs, the risks of these side effects can be severe to the patient's well-being. In the case of hernia surgery, the plastic mesh will always become situated in close contact with the sensitive intra-abdominal organs.

The progress within hernia repair mesh implant development, as well as in the development of mesh implants for the use of reconstruction of soft tissue defects in general, has been towards mesh implants with less mass in order to minimize foreign body reactions, and larger pore sizes, which on one hand reduce the mass of the mesh implant and on the other facilitate ingrowth of tissue. However, problems remain with such conventional mesh implants in addition to those noted herein.

Further problems with the various surgical procedures employing mesh implants are the time needed to unfold the mesh implant and time to attach it to the target location within the body cavity as well as the fasteners in the form of staples, tacks or the like used for implanting the mesh implants to the patient's tissue or organs. While other tissue fastening devices have been proposed which differ from staples per se, such other fasteners may have a plurality of components and other associated problems therein. For example, such fastening devices require access to both sides of a tissue site since they typically include an upper section having a crown and legs and a lower receiver, wherein the lower receiver engages and locks the legs of the upper section, making the surgical procedure longer and more complicated, resulting in potential complications to the patient. The act of positioning the folded mesh over the hernia defect is a major step in the Laparoscopic Ventral Hernia Repair. This segment of surgery can take up to 30% of the total surgical time. The task of unfolding and anchoring the edges of the mesh requires multiple trocars and instruments and skill by the surgeon to manipulate the instruments and the unfolded mesh implant within the body cavity, locating it over the target area, holding it in the position while at the same time fastening it to the target area within the body cavity. This extended surgical time can lead to complications in the patient while under anesthesia as well as complications after the surgical procedure.

Thus, there is a need to provide improved devices which can deploy a mesh implant which is relatively easier to employ. The mesh implant should be able to deploy in the area of the target hernia and is easily and quickly capable of fastening or attachment to the target area within the body cavity. Further, a need exists for a mesh implant that includes optional fastening means with are easy to apply and relatively difficult to remove once attached to the target area. In particular, there is a need for surgical fastening devices which do not require a second separate piece or receiver to lock, form or maintain the fastener in place in tissue, which can be applied and secured from one side of a tissue site, most desirably in an endoscopic or open surgical procedure, and which overcome the disadvantages associated with conventional surgical staples or tacks or sutures.

The area of robotic surgery also needs a device or apparatus employing a mesh implant which can be used for hernia surgery. In general, surgical devices are inserted into the abdominal cavity not through the umbilicus but through the fascia at a location where the laparoscopic device is below the target are of the hernia. The laparoscopic device will deploy a mesh implant but does not use stitches, tacks or fasteners but rather a surgical glue such as fibrin sealant. There are many complications with this method and using surgical glue based on lack of tackiness or securing the mesh implant to the target tissue. The better method is to use tacks or sutures, but current robotic devices and methods cannot employ such. This, a need exists for a surgical mesh deployment and implant device which can be used in robotic surgery to repair a hernia using tacks or sutures.

Further needs in the art include a mesh implant and mesh implant deployment device which stabilizes the area of surgery, deploys or activates the mesh implant, connects the deployed mesh implant to the tissue via conventional or inventive fasteners, and closes the incision point or port, with one device. The inventive mesh implant device may be used for a variety of surgical procedures, such as a hernia, but may also be used solely to close a surgical incision port or point, such as at the umbilicus or any incision point on a patient's fascia or within a body cavity or elsewhere. The inventive device may decrease surgical time, decrease the number of surgical instrument within the incision point, decrease the potential complications and decrease pain for the patient.

Accordingly, there remains a need in the art for the inventive mesh implant device including a mesh implant, its method of use and a kit containing the inventive device. The present invention provides a solution for these needs and other needs. The present invention has been made to solve the above problems occurring in the prior art and other needs in regard to surgical instruments and methods of treatment.

### SUMMARY OF THE INVENTION

In one aspect, a surgical instrument and assembly for laparoscopic procedures is provided, which is adapted and configured to include a mesh implant device, a mesh implant, supports for the mesh implant which activate to deploy the mesh implant within the body cavity and moved to placement over the target area with the supports removable upon securing the mesh to the target area.

In yet another aspect a surgical instrument and assembly for laparoscopic procedures is provided, which is adapted and configured to include a mesh implant device, a mesh implant, supports for the mesh implant which activate to deploy the mesh implant within the body cavity and moved to placement over the target area and a connection means attached to the deployed mesh implant to secure it to the target area, with the supports removable upon securement of the mesh implant to the target area, and a suture configured to close the incision point above the mesh implant once implanted during use.

In yet another aspect, a method for laparoscopic procedures is provided including use of a surgical instrument for laparoscopic procedures, which is adapted and configured to include a mesh implant device, a mesh implant, supports for the mesh implant, and an optional suture to close the incision point above the mesh implant.

In another aspect, a kit for laparoscopic procedures is provided including a device which is adapted and configured to include a mesh implant device, a mesh implant, supports for the mesh implant, and a suture to close the incision point above the mesh implant.

### DESCRIPTION OF THE DRAWINGS

The above and other advantages of the present invention will become readily apparent with reference to the following detailed description when considered in conjunction with the accompanying drawings which are incorporated in and constitute part of this specification are included to illustrate and provide a further understanding of the devices and related methods of the invention. Together with the description, the drawings serve to explain the principles of the invention, wherein:

FIG. 1 is a perspective view illustrating one embodiment of the inventive mesh implant device with the mesh implant retracted within the housing;

FIG. 2 is a perspective of a cut-away view the proximal end of the housing of FIG. 1 illustrating one embodiment of the inventive mesh implant device with the mesh implant retracted within the housing and showing the optional suture and proximal end of the supports exposed;

FIG. 3 is a further perspective of a cut-away view of FIG. 2 further including an optional stabilize device on the central portion of the housing of the inventive mesh implant device;

FIG. 4 is a perspective view of FIG. 1 of one embodiment of the inventive port closure device with the mesh implant partially activated and extending from the housing;

FIG. 5 is a full view of FIG. 1 of one embodiment of the inventive mesh implant device showing the partially activated mesh implant;

FIG. 6 is a close up view of a distal end of FIG. 1 of one embodiment of the partially activated mesh implant within the inventive mesh implant device wherein an optional fastener is a hook;

FIG. 7 is a view of one embodiment of the fully activated mesh implant having optional fasteners as hooks and optional suture in the inventive mesh implant device of FIG. 1;

FIG. 8 is a side cut-away view of FIG. 1 of one embodiment of the mesh implant device showing the distal end of the housing and the internal folded, inactivated and non-deployed mesh implant having optional fasteners.

FIG. 9 is a side cut-away view of another different embodiment of the mesh implant device showing the distal end of the housing and the internal folded, inactivated and non-deployed mesh implant having optional fasteners and including an optional stabilization device;

FIG. 10 is a side view of a cut-away exploded view of FIG. 1 of one embodiment of an activated mesh implant device with the mesh implant having optional fasteners partially activated and deployed out of the housing;

FIG. 11 is a perspective view of FIG. 1 of one embodiment of an activated mesh implant device with the mesh implant partially activated out of the housing and an optional stabilization device;

FIG. 12 is a top-downward view of another different embodiment of the inventive mesh implant device with the mesh implant having optional fasteners and an optional suture fully activated and including an optional stabilizing device on the housing, with the device within a patient's fascia or body cavity;

FIG. 13 is a side view of another different embodiment of the inventive mesh implant device with the mesh implant having optional fasteners and an optional suture partially activated;

FIG. 14 is a side view of FIG. 13 of one embodiment of the inventive mesh implant device in use within a patient's fascia or body cavity with the mesh implant fully activated perpendicular to the device itself and showing the suture;

FIG. 15 is a side perspective view of FIG. 1 of one embodiment of the present inventive mesh implant device in use within a patient's fascia or body cavity showing the mesh implant having optional fasteners fully activated, the housing and supports having been retracted outside of the patient's fascia or body cavity, and showing the optional suture;

FIG. 16 is a side view of FIG. 15 of one embodiment of the inventive mesh implant device in use within a patient's fascia or body cavity and with the mesh implant having optional fasteners fully activated and attached to the target tissue under the patient's fascia, and showing the optional suture;

FIG. 17 is a perspective view of another different embodiment of the inventive mesh implant device including a pistol grip activation means and the mesh implant folded and stored within the housing in a non-activated state;

FIGS. 18A and 18B are view of the inventive fastener to be used with the inventive mesh implant device of FIG. 1 or any other embodiments, with FIG. 18A a perspective view of one embodiment of an inventive fastener and FIG. 18B a side view of an inventive fastener;

FIG. 19 is a side view of another embodiment of the inventive mesh implant device including an inventive lever system and cone system with the mesh implant folded and stored within the housing in a non-activated state;

FIG. 20A is a cut away view of FIG. 19;

FIG. 20B is an exploded view of the lever system of FIGS. 19 and 20A;

FIGS. 21A-D are exploded view of the cone system including a side view of the top portion of the cone (21A), bottom view of FIG. 20A (21B), cut away view of the cone system including the supports (21C), side view of cone system including the supports (21D), and a top view of the cone system including the supports (21E);

FIG. 22A is an cut away side view of FIG. 19 with the mesh implant unfolded and out of the housing in an activated state;

FIG. 22B is a perspective view of FIG. 22A;

FIGS. 24A-G are further views of another embodiment of the method of the present invention of the mesh implant device of FIG. 19 starting with insertion into the umbilicus with the mesh implant in a non-activated state (23A), pushing on the deployment rod to deploy the mesh implant out of the housing but it has not opened up yet (23B), the mesh implant has activated and opened up into an umbrella configuration (23C), the deployment rod is pulled back out of the housing to pull the deployed umbrella mesh implant upward towards the housing (23D), the deployed activated umbrella mesh implant is placed against the hernia or target area within the body cavity and attached to it by sutures or tacks (23E), the control shaft for the supports is turned and the supports are pulled away from the mesh implant and back into the housing (23F), and the inventive device is removed from the body cavity through the umbilicus (23G); and

FIG. 24 is yet another embodiment of the present inventive mesh implant device including a mesh implant which is configured to be in the shape of an inverted umbrella when the mesh implant is in an activated state within the body cavity.

### DETAILED DESCRIPTION OF THE INVENTION

Reference will now be made in detail to the present invention, examples of which are illustrated in the accompanying drawings.

In accordance with one aspect of the invention, a mesh implant device is provided including an unactuated and non-deployed mesh implant and the device in one embodiment having the optional capacity to close the port or incision point through which the inventive device is inserted into the fascia, a body cavity or other location within a patient. In accordance with one aspect, the inventive mesh implant device has a housing with a folder and non-deployed mesh implant within the distal end of the housing. The mesh implant is configured similar to an umbrella including a plurality of supports. The support may be retracted at a proximal end of the housing upon connecting of the mesh implant via a connecting means outside of the inventive device to the tissue within the body cavity of the patient. In another embodiment the mesh may be connected to the fascia or tissue via sutures, stiches or tacks and the like. In another embodiment the mesh implant includes a plurality of fasteners on the outer edge of the mesh implant as a connecting means to attach the mesh implant to the fascia or tissue within the body cavity of the patient. In another embodiment, the mesh implant includes a suture so that upon connection of the deployed mesh implant and retraction of the supports and the housing, the main suture which is connected to the mesh implant (in one embodiment in the center of the mesh implant) is used to close the incision port. The device may include a deploying mechanism such as a push rod, pull rod, loop grip mechanism, thumb grip mechanism, pistol grip mechanism or other mechanism for the surgeon to deploy the mesh implant. Certain coring or removing of the material such as on the cone assembly (referenced as 900) are part of the design of the cone assembly 180 based on pricing and assembly such that the cone assembly 180 is lighter. Certain coring 900 is also shown in the deployment rod 190 and the support retraction rod or core shaft 169 but again are used during manufacture and assembly for pricing and weight of the final assembled inventive mesh implant device.

It should be noted that although the devices of the present invention are advantageous for various laparoscopic surgeries, including closing an umbilicus opening or for hernia procedures including robotic surgical procedures, they can advantageously be applied to close other incision ports or to deploy the mesh implant to other parts of the body, for example, within a uterine wall or the like.

The inventive mesh implant device advantageously provides one device through which a mesh implant may be inserted into the body cavity of a patient and deploy the mesh implant at a certain chosen location within a body with one device. Optionally the mesh implant may include inventive fasteners for reduction in the amount of time during surgery as well as reducing the number of instruments or devices within the body cavity at the same time. In yet another embodiment of the inventive mesh implant device, an option suture is included on the mesh implant used to close or suture the incision port again, using only one device thus reducing the surgical time and number of devices within the body cavity at the same time during the surgical procedure. The inventive mesh implant device thus may reduce surgical time, surgical costs, number of ports or incisions needed for the surgical procedure, as well as potentially reduce complications and pain for the patient.

Preferred embodiments of the subject invention are described below with reference to the accompanying drawings, in which like reference numerals represent the same or similar elements. One of ordinary skill in the art would appreciate that while the inventive device and mesh implant discussed herein relate to hernia surgical procedures within the abdominal body cavity, however the scope of the invention is not limited to those exemplary body parts and applications and may be sized and shaped for the anatomical portion for which a mesh implant may be needed.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials are now described. All publications mentioned herein are incorporated herein by reference to disclose and describe the methods and/or materials in connection with which the publications are cited.

It must be noted that as used herein and in the appended claims, the singular forms “a”, “an” and “the” include plural referents unless the context clearly dictates otherwise. Thus, for example, reference to “a stimulus” would include a plurality of such stimuli and reference to “the signal” would include reference to one or more signals and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may differ from the actual publication dates which may need to be independently confirmed.

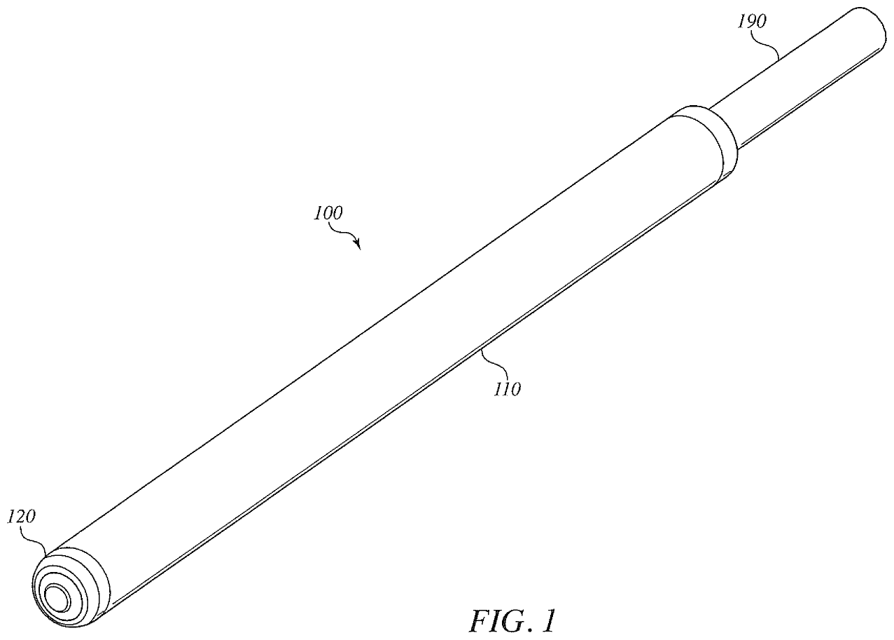

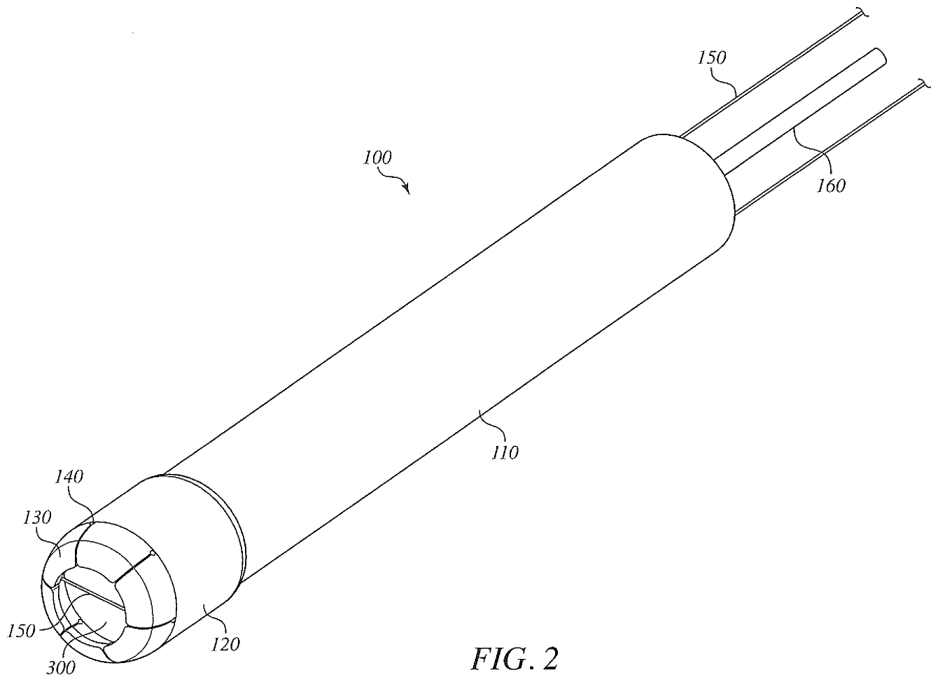

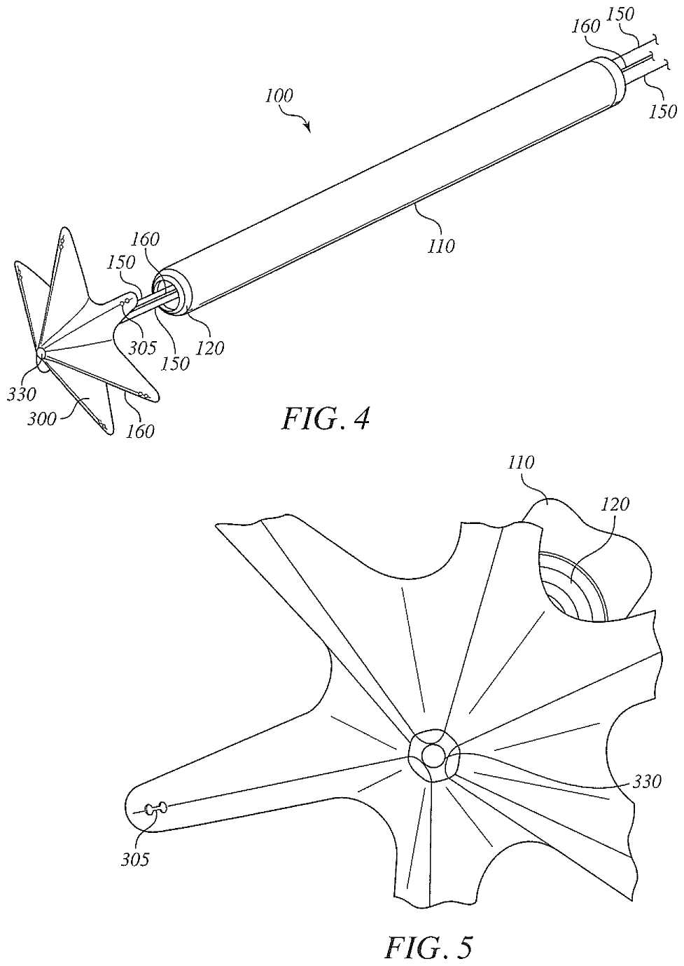

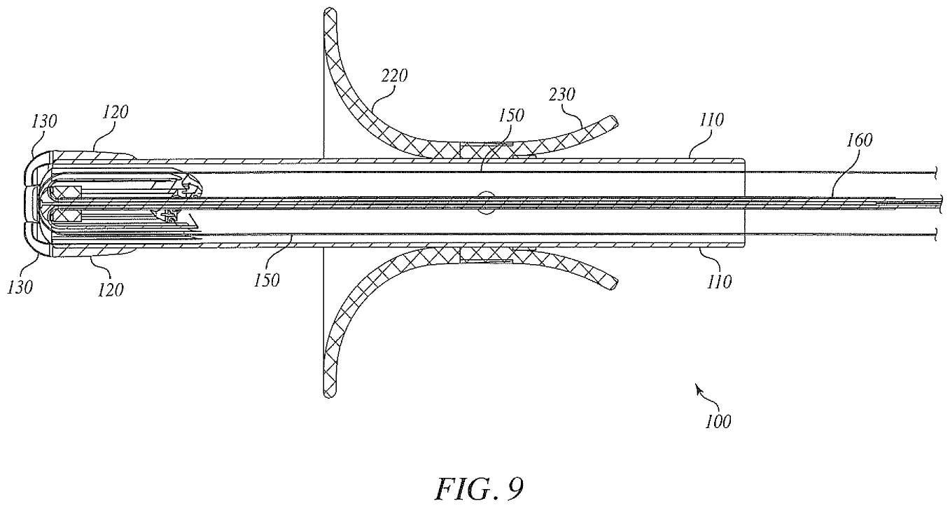

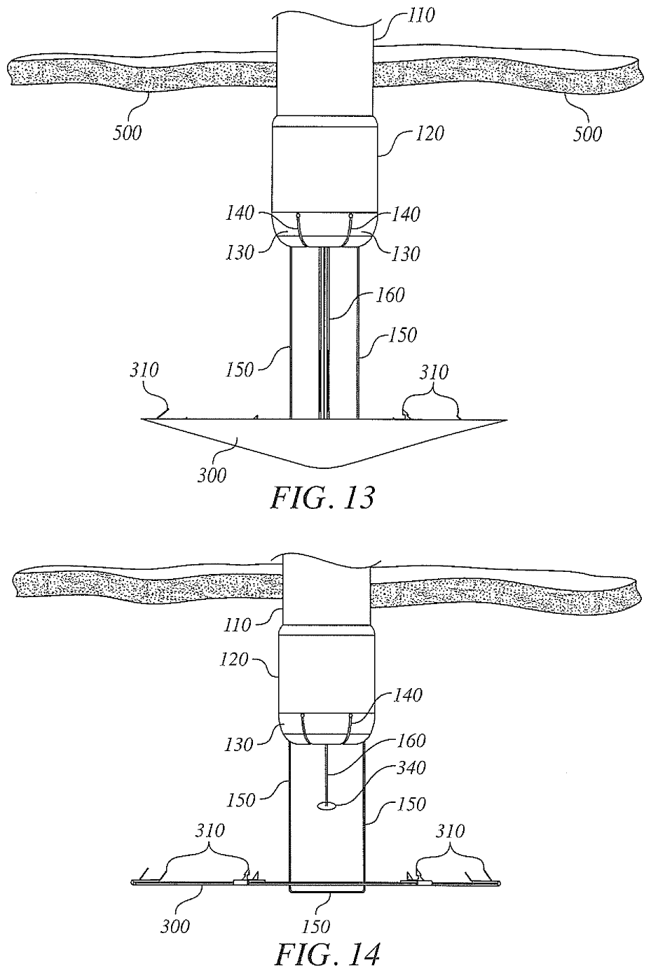

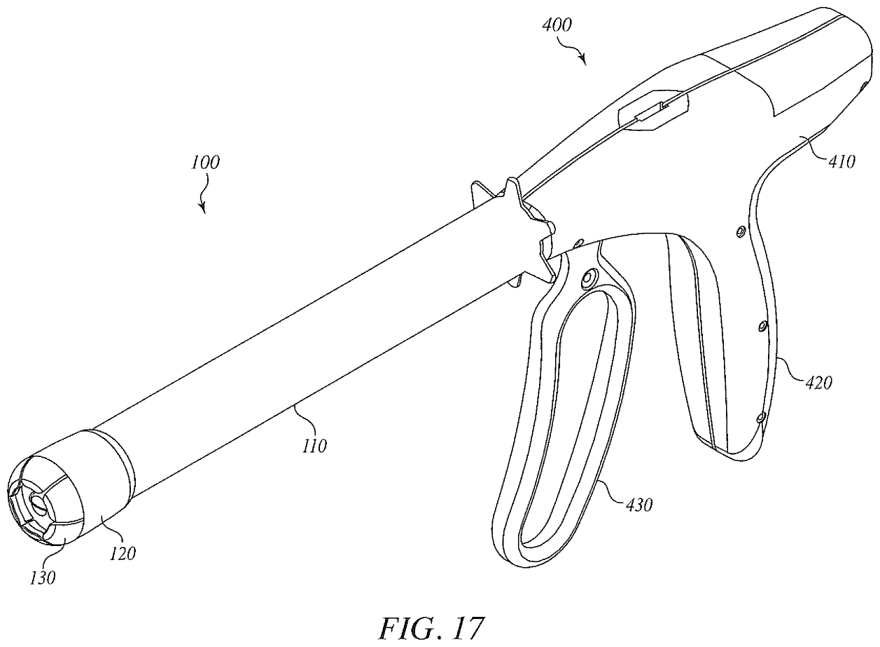

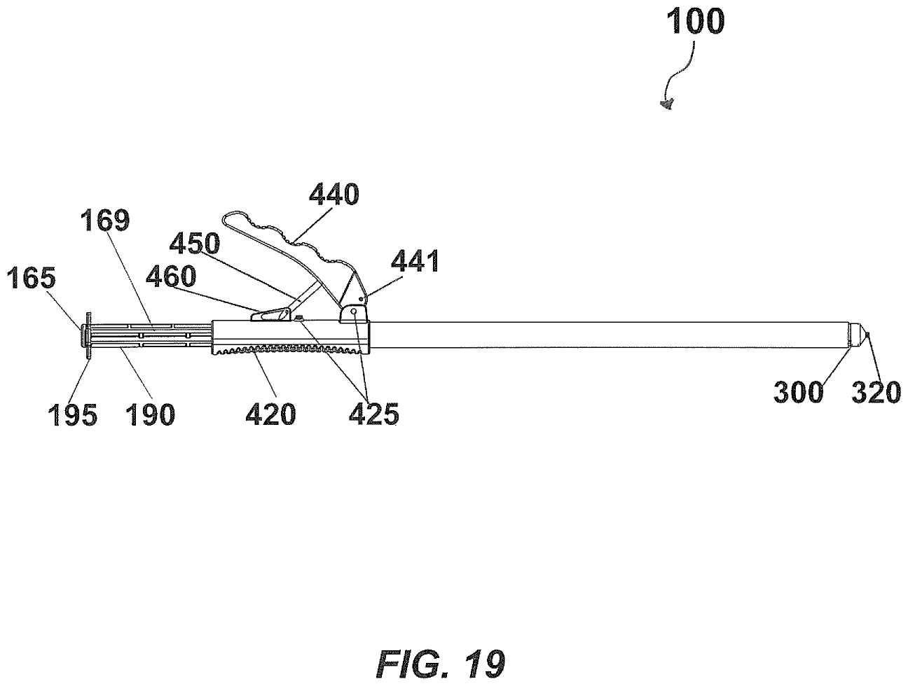

For the purposes of explanation and illustration, and not limitation, in accordance with the invention, an exemplary embodiment of a mesh implant device 100 in an unactuated position is illustrated in FIGS. 1 through 3 and in an actuated position is illustrated in FIGS. 2 and 4 through 16 with other embodiments shown in FIGS. 17-24. In accordance with these examples, the mesh implant device 100 includes a housing 110 having a diameter in a range of about 1 mm to about 50 mm, with a length of about 10 mm to about 1000 mm including in a range of about 20 mm to about 400 mm of length. The length of the housing 110 may be dependent on the target location for the mesh implant to be fastened within the body. The housing 110 may be a tube or cannula. The housing 110 may be hollow in which to hold the folded, unactuated and non-deployed mesh implant (300, not shown in FIGS. 1 and 19) at the distal end of the housing 110 and at the proximal end a deployment mechanism 190 for the mesh implant. In one embodiment the diameter of the housing 110 is about 7.5 mm as shown in FIG. 1 whereas the diameter is about 27 mm as shown in FIGS. 19-24, with other embodiments including but not limited to a housing 110 diameter of about 2 mm, 3 mm, 5 mm, 10 mm, 12.5 mm, 15 mm, 16 mm, 20 mm, 25 mm, 30 mm, 35 mm, 40 mm, 45 mm, 50 mm or larger. The diameter of the housing 110 may be related to the dimensions of the folded unactuated mesh implant (300) as within the inner diameter of the hollow housing 110. For instance, a pediatric patient may require a smaller diameter deployed and actuated mesh implant 300 such that the diameter of the housing 110 may be about 3 mm or about 5 mm whereas other patients may require larger diameters. Further, the diameters of the housing 110 may be affected by the size of the mesh implant 300 as seen by the differences of the mesh implant 300 sizes in FIGS. 4-7 and 11-16 compared to the size in FIGS. 19-24.

Housing 110 may be comprised of any material compatible to the human body as the distal end of the housing 110 will be placed within the body cavity for deployment of the mesh implant 300. Such materials for the housing 110 include plastics or metals. If a metal, the housing 110 may be comprised of stainless steel and may further be coated with a shrink wrap plastic such as shrinkable polyethylene fiberglass, or polyvinyl chloride of a grade suitable for use in surgical procedures. The housing 110 may be comprised of many known polymers such as polycarbonates or ABS. For cost effectiveness, the housing 110 may be comprised of any plastic capable of sterilization according to regulatory agencies for medical devices. Typically the housing 110 will be a single use sterilized material but it is envisioned that other materials may be used that can be re-sterilized and the inventive mesh implant device 100 could be reusable.

The dimensions of the housing 110 may be a diameter in the range of about 1 mm to about 50 mm preferably between about 3 mm to about 35 mm. The dimensions of the housing 110 may also be such that the diameter is slightly larger or smaller than the maximum diameter of the unfolded, deployed, actuated and open mesh implant 300. In an embodiment not shown the housing 110 can include printed lines which can be used by the surgeon or medical provider to ascertain the depth of the housing 110 when inserted within the fascia 500 of the patient. The housing 100 may further include an orientation line printed perpendicular to the distal end of the mesh implant 300 to indicate orientation of the width of the mesh as well as orientation of the distal end of the optional suture 150. Other indicators may be envisioned and added to assist the surgeon during the procedure. In another embodiment the housing 110 has a slot.

The housing 110 may be hollow and thus includes a channel which houses the folded non-deployed unactuated mesh implant 300 which mesh implant 300 is connected to the deployment mechanism 190. The deployment mechanism may be a push rod 190 as shown in FIGS. 1 and 19-24 with a grip 195 on the proximal end, or a pistol grip deployment mechanism 400 as shown in FIG. 17 or may be any known conventional deployment mechanism. In an unactuated position, the deployment mechanism 190, in this embodiment being a push rod 190 has a distal end partially housed within the housing 110 and a proximal end extending out of the proximal end of the housing 110 which in some embodiment including a grip 195 for use by the surgeon or medical professional. To deploy the folded unactuated mesh implant 300 the rod 190 is pushed in a downward direction toward the distal end of the housing 110 such that the rod 190 resides folly within the housing 110 or partially resides within and with a portion of the proximal end of the rod 190 extending out of the housing 110. In the actuated position the mesh implant 300 is moved from a stowed position within the distal end of the housing 110 to a deployed position by pushing the mesh implant 300 out of the housing 110. The rod 190 may be connected to a rod grip which the surgeon may use in actuating the inventive device 100, such as a ring grip, finger grip, thumb grip, pistol grip, or other known grips. As shown in FIGS. I 9-24, the proximal end of the deployment rod 190 includes a knob or grip 195 for better grip and ease of movement of the deployment rod 190 by the surgeon or other medical professional when in use during a surgical procedure such as a hernia repair operation.

In another embodiment of the present invention as shown in FIG. 17 the mesh implant device 100 includes a pistol grip deployment mechanism 400. The pistol grip deployment mechanism 400 includes a handle 410 with internal mechanisms to deploy the folded unactuated mesh implant 300. The handle 410 includes a trigger 430 and a proximal housing member 420. Other deployment mechanisms 190 may be employed.

The distal end of the housing as shown in FIGS. 1-17 includes a cap 120, which is not included on the embodiment of the present inventive mesh implant device 100 shown in FIGS. 19-24 which instead include a cone 180 below the mesh 300. The cap 120 has a distal end 130 which may be tapered and may be solid or may include optionally a plurality of indentations 140 to facilitate the deployment of the supports 160 connected to the mesh implant 300. For instance, and by way of example only, if the mesh implant device 100 includes six supports 160 connected to the mesh implant 300 then there may be six corresponding indentations 140 within the distal end 130 of the cap 120. The cap 120 may be of any material compatible to the human body as it will be placed within the body cavity for deployment of the mesh implant 300. Such materials for the cap 120 include plastics or metals. If a metal, the cap 120 may be comprised of stainless steel and may further be coated with a shrink wrap plastic such as shrinkable polyethylene fiberglass, or polyvinyl chloride of a grade suitable for use in surgical procedures. The cap 120 may be comprised of many known polymers such as polycarbonates or ABS. For cost effectiveness, the cap 120 may be comprised of any plastic capable of sterilization according to regulatory agencies for medical devices.

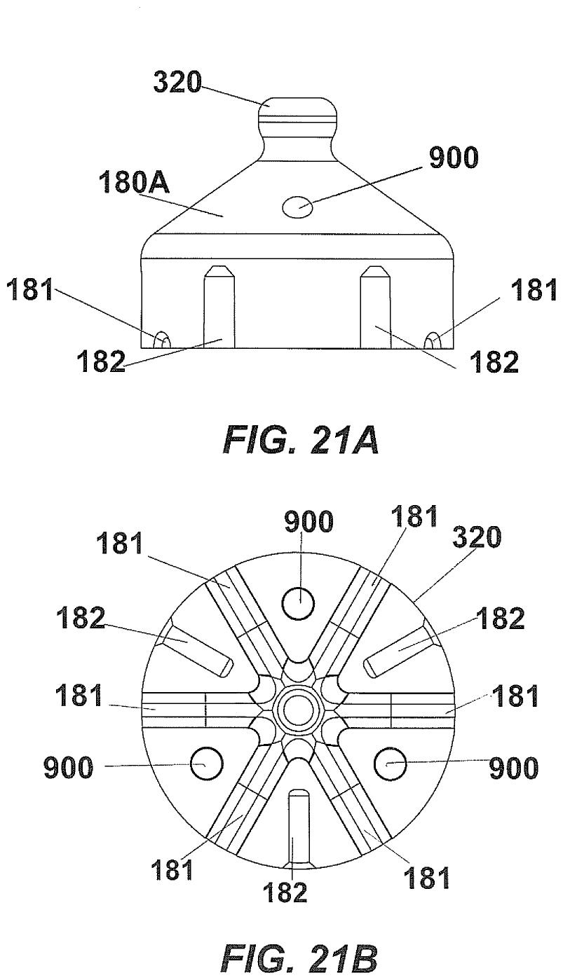

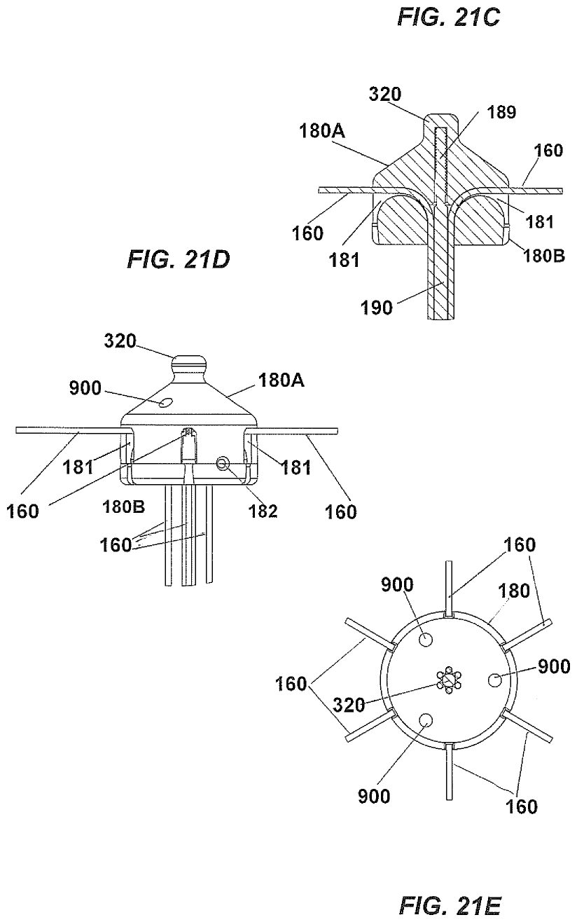

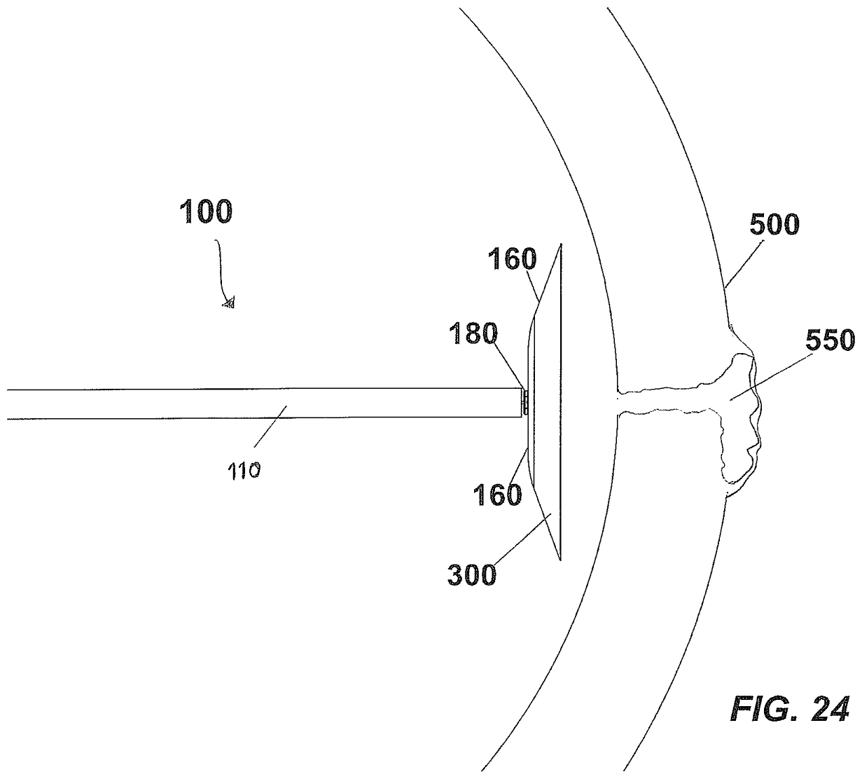

In the embodiments as shown in FIGS. 19-24 a cone assembly 180 is attached on the distal end of the deployment mechanism 190 and under the center point of the mesh implant 300. The cone assembly 180 includes a distal end extension cap 320 which pushes through the center of the mesh implant 300 to connect the cone assembly 180 to the mesh implant 300. If there are by way of non-limitation example only, six supports 160 connected the mesh implant 300, the cone assembly 180 includes six corresponding indentations 181 within the connection of the upper portion 180A and the lower portion 180B of the cone assembly 180 as shown in FIGS. 21A-E, though in this embodiment as shown the curve of the indentations or apertures 181 within the cone assembly 180 are at a slightly rounded 90 degree-right angle as the mesh implant 300 would then deploy and open up in the configuration of a flat umbrella the angle of the indentations or apertures 181 shown in FIG. 21C, but in the embodiment shown in FIG. 24, the supports 160 of the deployed and activated mesh implant 300 are in a different inverted angle and as such the indentations 181 within the cone assembly 180 would have to be at a higher angle in FIG. 21C (this embodiment is not shown) whereas the supports 160 would not exit the cone assembly 180 at a right angle on the perimeter of the cone assembly 180, but would exit the cone assembly 180 on the upper potion 180A of the cone assembly 180 such that the shape configuration of the mesh implant 300 is of an inverted umbrella like when the wind turns the umbrella inside out and in an upward configuration. As an example and by way of no-limiting design, after the bottom portion 180B is connected, such as by adhesion or glue in the glue indentations 182, to the upper portion 180A, the supports 160 are slightly advanced to pull the bend past the cone 180 base to eliminate this initial force spike when closing the lever 440 on the first stroke. The shape of the deployed and actuated mesh implant 300 into the umbrella shape, inverted or not, is controlled by the relief angle in the indentations or apertures 181 within the cone assembly 180. Whatever the chosen configuration the supports 160 would be the same but the angle of the indentations 181 for insertion and extension upon deployment and activation of the supports 160 would be different within each cone assembly 180 and any varied angles could be used from 5 degrees to 189 degrees.

The cone assembly 180 in the embodiments shown in FIGS. 19-24 is comprised of two portions, a bottom portion 180 and an upper portion 180A, which in this non-limiting embodiment are connected via an adhesive such as glue placed within glue indentations 182 though other connection means may be employed such as welding and the like. The areas where the bottom portion 180B are joined to the upper portion 180A also in this embodiment include indentations or apertures 181 for the supports 160. The supports 160 may be super elastic nitinol wires are used to deploy the mesh implant 300. The mesh implant 300 with the interwoven supports 160 is inserted into the back or proximal end of the housing 110 such as an outer tube during the assembly operation of the inventive mesh implant device 100. This causes the mesh implant 300 with the supports 160 to fold and pleat to fit into the inside diameter of the housing 110, and a funnel type insertion fixture with grooves matched to the supports 160 positions guides the folds in the mesh implant 300 to be uniform or more or less even, though other configurations could be employed. During insertion each supports 160 wraps around a corresponding molded plastic tab with tangent external radii that are large enough to prevent the yield stresses in the corresponding supports 160 diameter from being exceeded. This assures that the unyielded supports 160 will elastically spring open and drive the mesh implant 300 to a fully deployed and predictable state of configuration when activated, such as an umbrella. The supports 160 are further constrained on their sides by the indentations or apertures 181 in the cone assembly 180 that mate with the cone base tabs with sufficient clearance to accept each supports 160 diameter. The supports 160 typically have free state forms of approximately 90 degrees to aide in assembly of the cone 180 with the indentations or apertures 181 between the upper portion 180A and the bottom portion 180B. This allows these two parts to be permanently fastened together without having any assembly forces resulting from forcing straight supports 160 or wires around the arced cone assembly indentations or apertures 181 by driving the cone assembly parts 180A, 180B together. This is especially useful when adhesive bonding is used in the glue indentations or apertures 182 as the upper potion 180A and the bottom portion 180B do not need to be held clamped together with special fixtures during the adhesive cure time. The supports 1160 are then slightly advanced at assembly to eliminate the initial force spike of pulling the forms in the supports 160 out of the cone assembly 180. This significantly reduces the initial hand force applied at the lever 440 to start support 160 retraction to release the mesh implant 300 from the inventive mesh implant device 100. Once the supports 160 are deployed and advanced out of the distal end of the housing 110, they spring open and deploy the mesh implant 300 in an attempt to return to a lower state of stress. As shown in FIG. 23B the mesh implant 300 is being advanced and deployed out of the housing 100 and within about a tenth of a second the supports 160 spring into the non-limiting example as shown in FIG. 23C. The angle of the support 160 deployment is defined by the relief angle of the indentations or apertures 181 molded in the cone assembly 180. Alternate relief angles besides the 90 degree angle shown in FIGS. 19-24 can be molded to provide device designs for deploying concaved or convex mesh implant shapes once the support 160 and folded mesh implant 300 are deployed and advanced out of the housing 110. This is useful for appropriately shaping the deployed mesh implant 300 for positioning and tacking when the inventive mesh implant device 100 is inserted through the fascia 500 near the target hernia defect 550 such as the umbilicus 510 or when approaching the defect from above when the inventive mesh implant device 100 is inserted through an alternate port.

The mesh implant 300 is folded within the distal end of the housing 110. The mesh implant 300 may be of any shape such as a circle, oval, sphere, rectangle, square, triangle, or the like. The mesh implant 300 may be of a size ranging from about less than 1 mm to about 500 mm on each side if non-spherical depending on the shape of the mesh implant 300. If in a spherical shape the mesh implant 300 may have a diameter of about less than 1 mm to about 300 mm. The shape, area and diameter of the mesh implant 300 may be dependent on the target tissue to be connected to the mesh implant 300 such as connecting to a hernia location with an area of about 1000 mm² or where there is the optional suture 150 then closing a port having a diameter of about 10 mm to about 40 mm. For instance, in one embodiment of the present invention not shown, the mesh implant 300 has a rectangular shape with the dimensions of about 150 mm and about 200 mm, though other dimensions and shape may be employed. The mesh implant 300 may be in the shape of a square, rectangle, circle, oval, triangle or any other shape configured to close the opening or port in the fascia 500 of the patient's body. In one embodiment of the present invention, the mesh implant 300 when fully activated is in the shape of a circle with six supports 160.

The mesh implant 300 may be comprised of any material compatible to the human body as it will be implanted within the body cavity. Such materials for the mesh implant 300 include plastics or metals. The mesh implant 300 may be comprised of many known polymers such as polycarbonates or ABS. In one embodiment of the present invention the mesh implant 300 is comprised of a surgical grade polyester or polypropylene mesh. Optionally, the mesh implant 300 may be coated or laminated on the distal side with a non-adhesion material such as without limitation a perforated LDPE film to reduce the potential for bowel adhesion or a progressively biodegradable material. Further, the mesh implant 300 may be a woven or knitted structure or can have a non-woven, for instance electro-spun, structure, wherein the (electro-spun) non-woven structure can further be furnished with man made through and through holes. When two or more materials are incorporated with each other, fibers of said materials, respectively, can be jointly woven, knitted or non-woven into the same suitable structure. Also, various materials can be spun into fibers which are braided, twisted into a multifilament produced from two or more materials, which multifilament is woven, knitted or non-woven into said suitable structure. It is understood that any combination of fibers in the form of monofilament, filament bundles, multifilament or braided or twisted multifilament can be combined into the desired structure.

The mesh implant 300 may further comprise bioactive or therapeutic substances including without limitation those naturally present in humans or of foreign origin. These substances include, but are not limited to, proteins, polypeptides, peptides, nucleic acids, carbohydrates, lipids or any combinations thereof. Especially considered are growth factors, such as PDGF, TGF or FGF, or components of the naturally occurring extracellular matrix, including cytokines, fibronectins, couagens, and proteoglycans such as but not limited to hyaluronic acid. Therapeutic substances and medicated coatings that are considered include, but are not limited to antibiotic drugs and pain relieving substances. Bioactive or therapeutic substances of human or foreign origin can be coated onto the mesh implant 300 or entrapped within the porous structure of the implant or incorporated through covalent or other chemical or physical bonding, in an active state or as precursors to be activated upon any physical or chemical stimuli or modification.

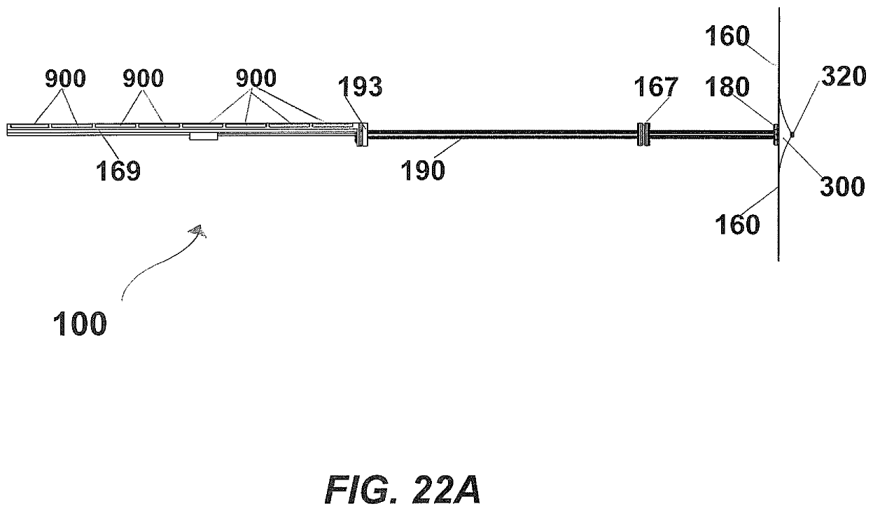

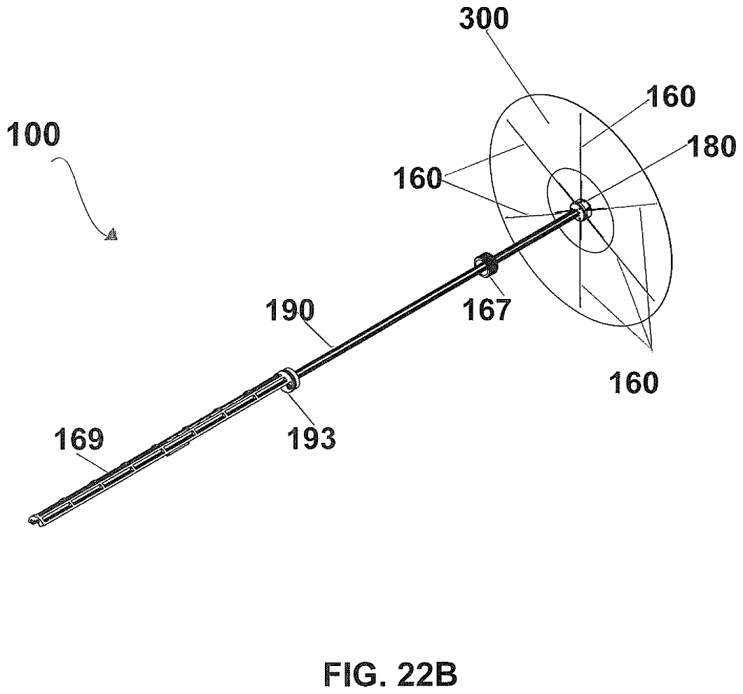

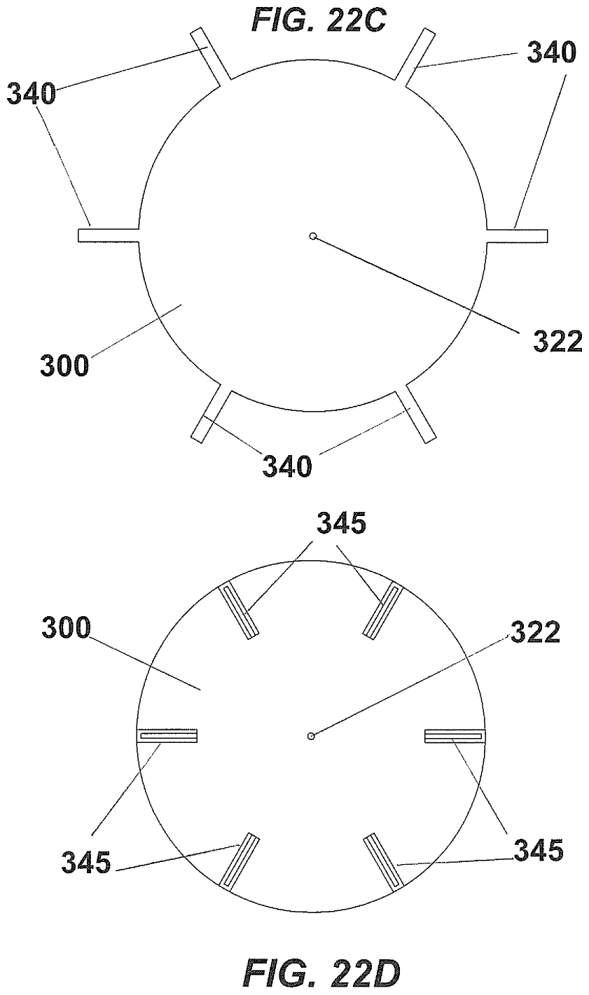

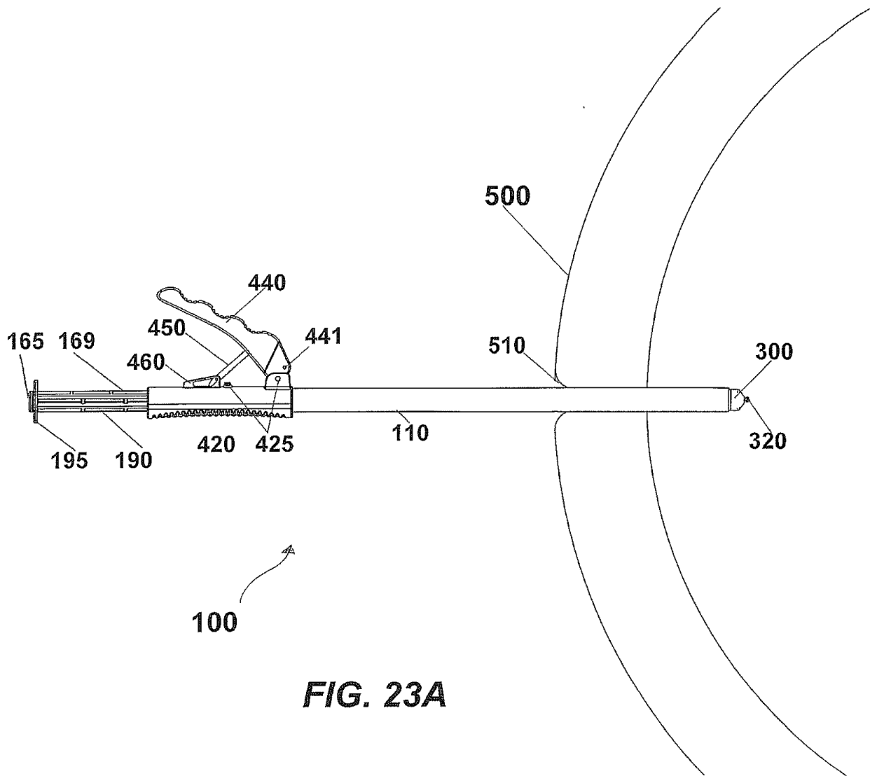

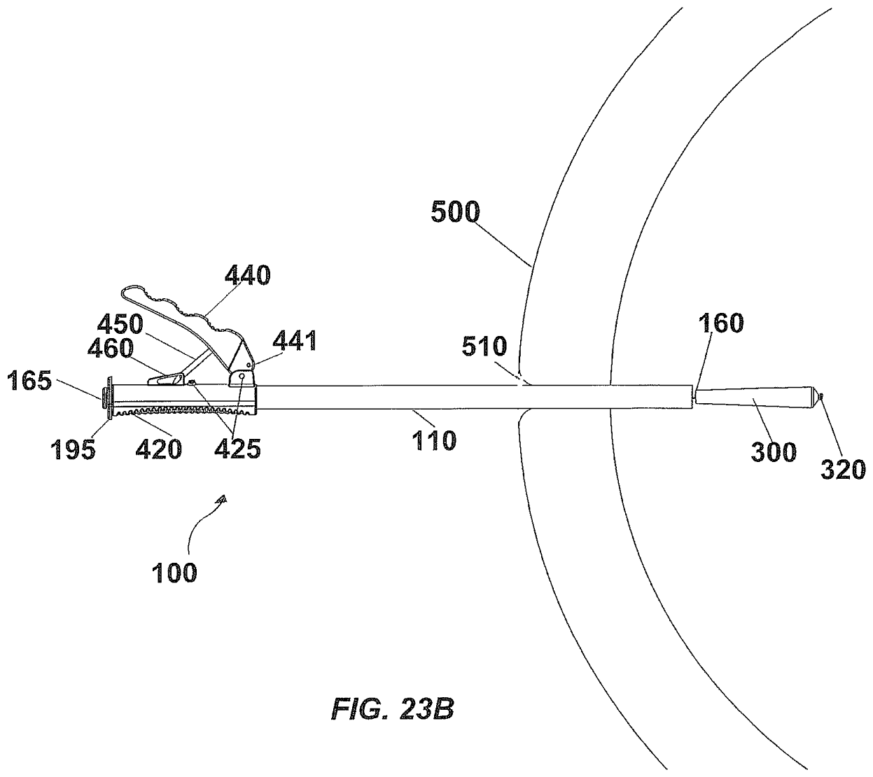

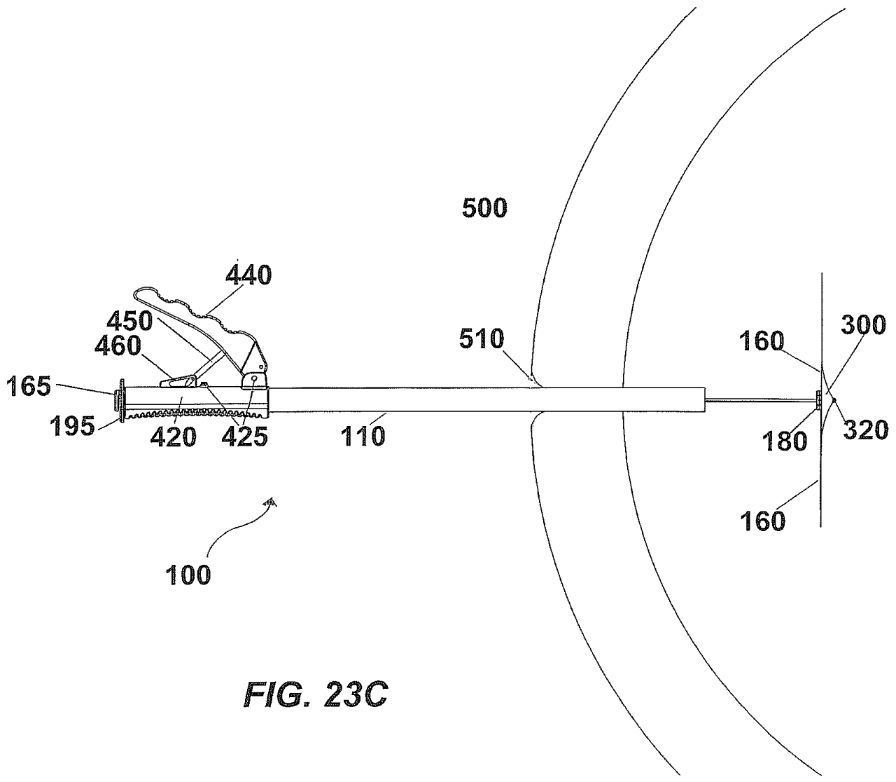

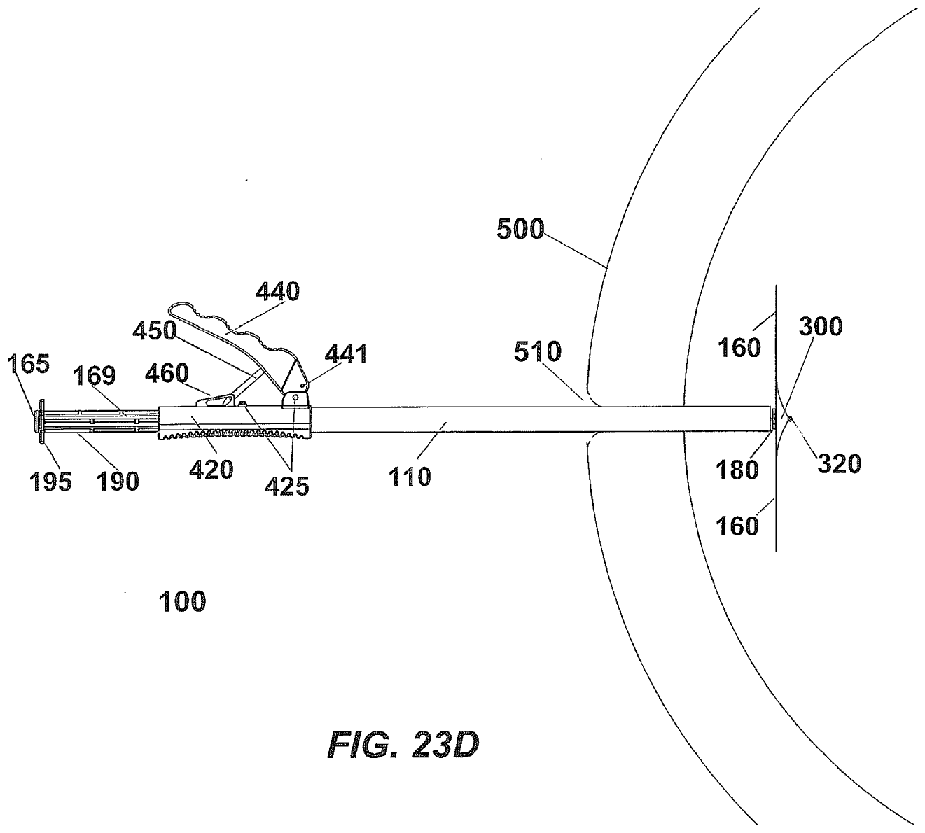

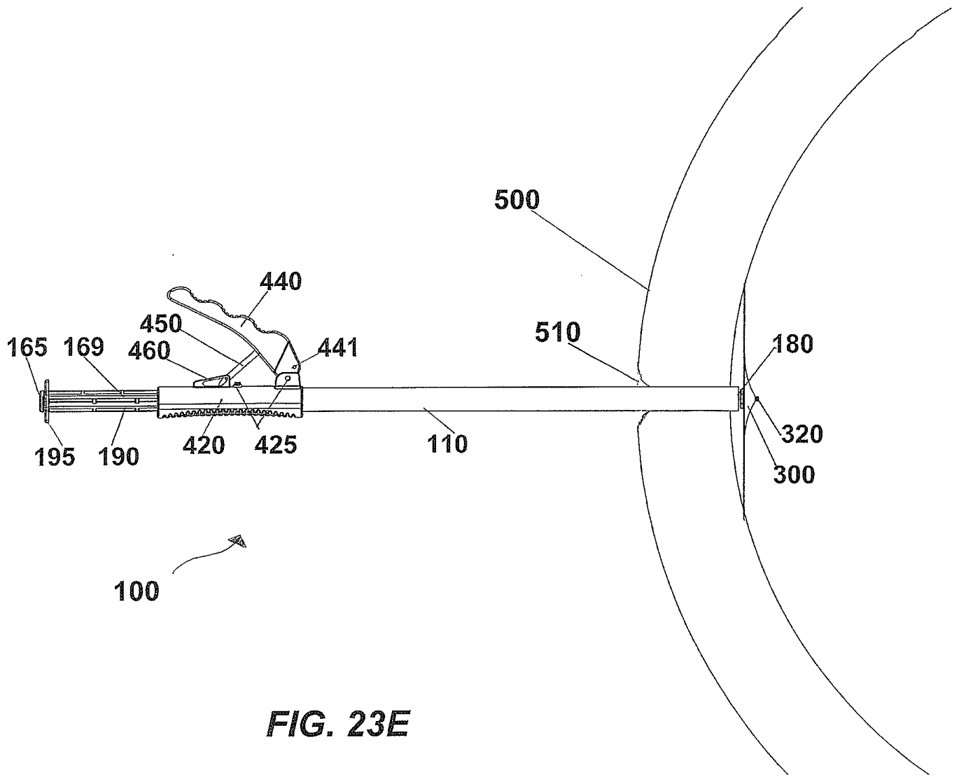

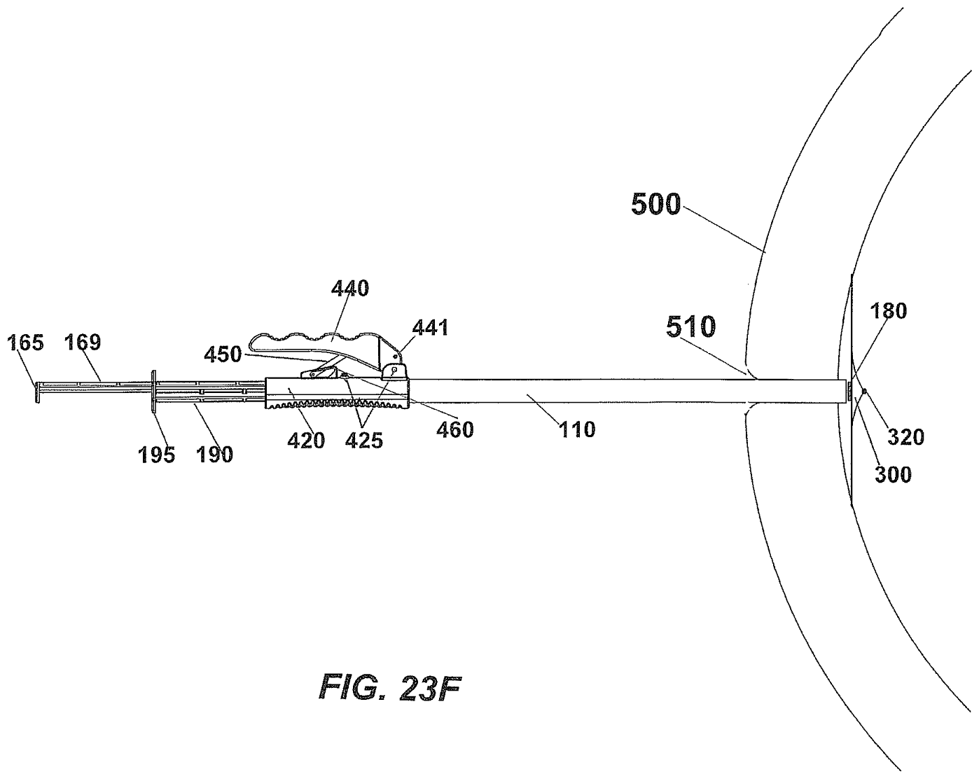

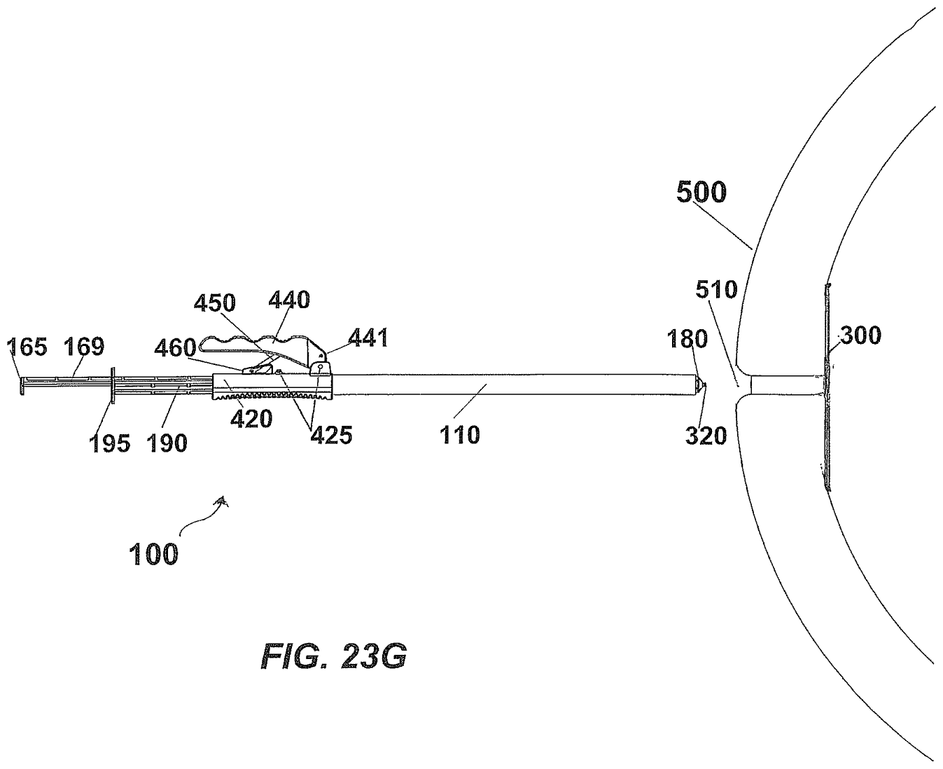

In one embodiment of the present invention as shown in FIGS. 1-24, the mesh implant 300 is in the shape of a circle. The mesh implant 300 is manufactured with a number of extensions 340 corresponding to the number of supports 160. As shown in FIG. 22C, by way of non-limiting example only, there are six extensions 340 for six supports 160. Each of the extensions are then folded back towards the center of the mesh implant 300 and connected on two sides but not the third edge to create a pocket 345 within which the distal end of each support 160 is inserted. The extensions 340 may welded on the two side, adhered or any other known connecting means to create a pocket 345 or opening in which the distal end of the support 160 may be inserted. The center of the mesh implant 300 further includes a hole 322 through which the cap 320 on the top of the cone assembly 180 is inserted to connect the cone assembly 180 to the mesh implant 300.

In one embodiment of the present invention the mesh implant 300 is folded within the distal end of the housing 110 in a shape similar to that of a folded umbrella. In this embodiment of the present invention the mesh implant 300 includes a plurality of supports 160 which on a distal end of each support 160 for example only, forms tines, ribs or barbs such as those on the underside of an umbrella. The supports 160 may number between 2 to 10 or more depending on the area of the tissue or organ to which the mesh implant 300 will be connected as well as the size of the mesh implant 300. In one embodiment of the present invention between about 4 to about 8 supports 160 are included to support the deployed mesh implant 300 in its actuated state as shown in FIGS. 4-7 and 10-16 as well as FIGS. 19-24. The supports 160 may be comprised any material compatible to the human body as it will be placed within the body cavity upon deployment of the mesh implant 300. Such materials for the supports 160 include plastics or metals. If a metal, the supports 160 may be comprised of stainless steel or titanium and may further optionally be coated with a shrink wrap plastic such as shrinkable polyethylene fiberglass, or polyvinyl chloride of a grade suitable for use in surgical procedures. The supports 160 may be comprised of many known polymers such as polycarbonates or ABS. For cost effectiveness, the support 160 may be comprised of any plastic capable of sterilization according to regulatory agencies for medical devices. In one embodiment of the present invention the supports 160 are comprised of nitinol and once released from the housing 110 spring out into a right angle to the housing 110 such that the mesh implant 300 is in a fully opened and activated state. In another embodiment of the present invention, the supports 160 are comprised of stainless steel such that once released from the housing 110 they spring out into a partial activated state relative to the housing 110 in the range of about 20 degrees to about 80 degrees with the mesh implant 300 in a partially opened and activated state so that the mesh implant in this partial activated and opened state can then be positioned within the body cavity to the target tissue location where the hernia 550 is located and in this embodiment the push rod 190 is then further moved in a distal direction so as to fully open the supports 160 and the mesh implant 300 to either a right angle of about 90 degrees or even in an inverted shape such that the mesh implant 300 would take the shape of an inverted umbrella (as shown in FIG. 24) and the support 160 is at an angle compared to the housing 110 of more than 90 degrees as shown in FIG. 24. This embodiment with an inverted umbrella activated state may be useful in robotic surgery where the inventive mesh implant device 100 is not inserted within the umbilicus but rather inserted in a different location around the abdomen and then the mesh implant 300 is activated partially within the body cavity and then moved to the location of the target tissue or hernia and at that time is then fully activated into a right angle or even inverted.

In an alternate embodiment the supports 160 are comprised of spring temper stainless steel wires as opposed to nitinol wires to deploy and hold the mesh implant 300. The spring temper stainless steel has a lower yield stress than the nitinol alloy so with similar wire diameters the stainless wires as supports 160 would take a set when flexed and assembled into the housing 110 or tube. Then when deployed from the housing 110 or tube the supports 160 or wires would not fully open the mesh implant 300 but only slightly open it such as an angle of about 10 degrees to about 80 degrees compared to the housing 110. The mesh implant 300 and supports 160 or wires are then pulled up to the distal end of the housing 110, where optionally there is a plastic ring that is fixed to the end of the housing 110 or tube. The rear knob or grip 195 on the push rod 190 is then rotated to further pull the support 160 or wires into the housing 110 and further open the mesh implant 300 to the desired angle such as about 90 degrees to about 170 degrees depending on the desired configuration such as right angle to the housing 110 or inverted to the housing 110 such that the angle of the fully deployed mesh implant 300 is 190 degrees of more compared to the perpendicular right angle of the deployment rod 190. The mesh implant 300 can be fully opened so that it is held flat by the supports 160 or wires, opened less so that it has a slight conical shape, or opened more than fully so that it has an inverted conical shape with any level of angle or shape desired by the surgeon when in use as shown in FIG. 24. This embodiment has the potential advantage of a more controlled and less rapid full mesh implant 300 opening within the body cavity and could also be used in robotic surgery. The surgeon can dial in the precise mesh implant 300 shape to closely approximate the target tissue curvature. The inventive mesh implant device 100 can be introduced through the hernia defect where the mesh implant 300 is tacked through the outer mesh surface or introduced through an incision opposite the hernia and tacked through the inner mesh surface such as in robotic surgery. The stainless wires have the advantage of higher stiffness than the same diameter nitinol wires which will help in the control and positioning of the mesh implant 300.

The distal end of each of the supports 160 is connected to the outer edge of the mesh implant 300 via a connecting means. The connecting means may be an pocket 345 as shown in FIGS. 22C-D which may also include optional adhesive within the pocket 345, or an adhesive alone or connected by a staple, crimp (ash shown in FIGS. 1-18), nut, bolt, screw, solder, tack, suture, glue or any known fastener. The connecting means may be biodegradable or non-biodegradable depending on the location within the body and proposed use for the mesh implant. The connecting means may be temporary in that the supports 160 will be removed from the mesh implant, either after implantation of the mesh implant 300 on the target tissue or at some later time during the same surgery or even a subsequent surgery after a period of hours or days or weeks or even later, or may be permanently connected to the mesh implant 300. In the embodiments shown in FIGS. 19-24, the mesh implant 300 is designed with corresponding radial tabs or pockets 345 for each support 160. Each tab or extension 340 may be folded back and welded, typically by ultrasonics or RF welding, to the base material around three sides keeping one side open for insertion of the distal end of the support 160. This creates a sleeve or pocket 345 that the end of each support 160 is inserted into. In one embodiment the supports 160 are woven several times through the openings between the woven mesh strands to hold the mesh to the supports 160. The distal end of the support 160 is designed to have a slight radius at the tip to assure that they are atraumatic and cannot snag a mesh strand, or patient tissue when the support 160 is detached from the mesh implant 300 and retracted back into the housing 110. After being woven through the mesh implant 300 each support 160 end is inserted into its welded sleeve or pocket 345 and the solid welded end of the sleeve or pocket 345 at the mesh implant's 300 outer perimeter constrains the support 1 60 in the sleeve or pocket 345 and prevents the support 160 from advancing past the perimeter of the mesh implant 300. A center hole or aperture 322 in the mesh implant 300 is slightly smaller than the undercut post or cap 320 on the top of the cone assembly 180 such that the mesh implant 300 stretches over the cap 320 to hold the mesh implant 300 centered during deployment and moving the folder mesh implant 300 within the housing 110 out into the body cavity of the patient as well as when the inventive mesh implant device 100 is moved within the body cavity and the activated actuated mesh implant 300 is fastened to the target tissue or hernia 550. After the mesh implant 300 is fastened such as through sutures, tacks, hooks, crimps, or other known fasteners in place over the target tissue or hernia 550, the lever 440 is cycled a number of times, such as by way of non-limiting example only six or seven times, and the supports 160 radially retract from their sleeves or pockets 345 and the points where they are interwoven through the mesh implant 300. The supports 160 arc pulled below flush with the cone assembly 180 outer diameter and release the mesh implant 300 from the device. The mesh implant 300 is now only attached to the undercut or cap 320 on the cone assembly 180 which disengages when the inventive mesh implant device 100 is removed, based on a tug or sufficient force to decouple the cap 320 out of the apertures 322. The inventive mesh implant device 100 is removed from the body cavity and the mesh implant 300 remains within the body cavity fastened or attached to the target tissue or hernia 550.

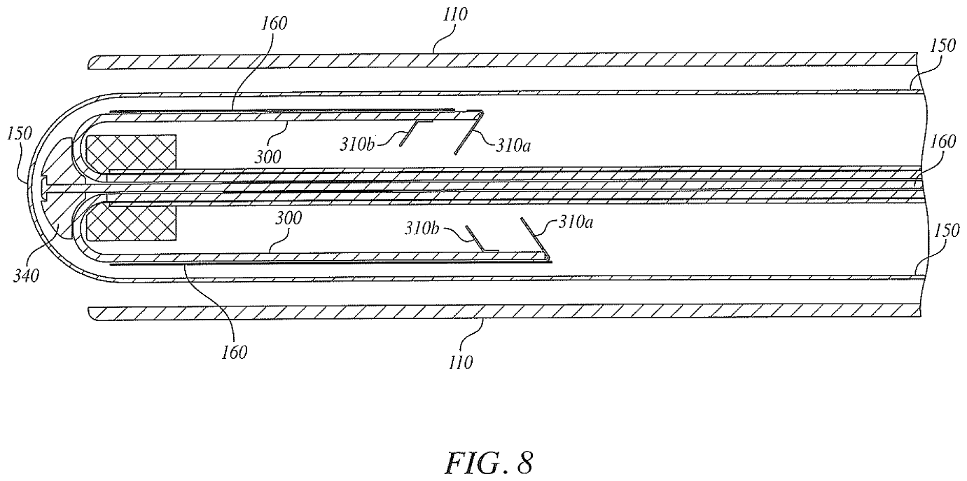

As shown in FIGS. 4-6, in each of these embodiments of the present invention a crimp 305 connects the supports 160 to the mesh implant 300. In another embodiment of the present invention as shown in FIGS. 8-11 an inventive fastener 310 connects the supports 160 to the mesh implant 300 and can also be used to fasten the mesh implant 300 to the target tissue. In yet another embodiment of the present invention the distal end of the supports 160 may be each alternately fastened to the mesh implant 300 in that every other support 160, for instance every even support 160 is connected to the mesh implant 300 whereas the other odd supports 160 are not connected at the distal end to the mesh implant 300. In another embodiment of the present invention as shown in FIGS. 22C-D, the distal end of the support 160 may be connected to the mesh implant 300 via a small cap 320 inserted into the center hole or aperture 322. In yet another embedment as shown in FIGS. 22C-D the supports 160 are connected to the outer edges of the mesh implant via pockets 345 where the distal end of the curbed portion of the support 160 is inserted into the pocket 345, or could then be inserted and sufficiently adhered such as by a biocompatible glue or adhesion where the support 160 stays within the pocket 345 during deployment but is detachable and removeable with a set force via the knob 165 and withdrawal of the rod 190 after the mesh implant 300 has been attached to the target hernia, fascia or body part.

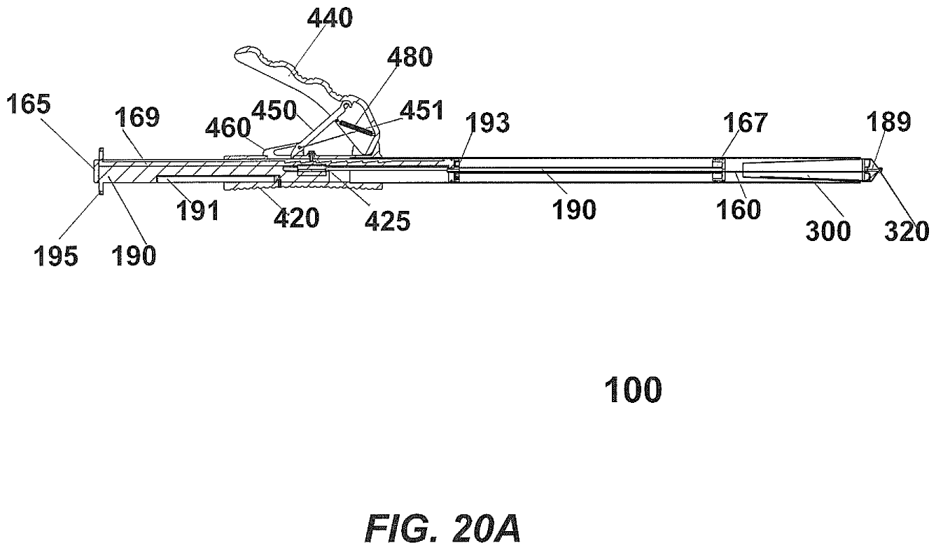

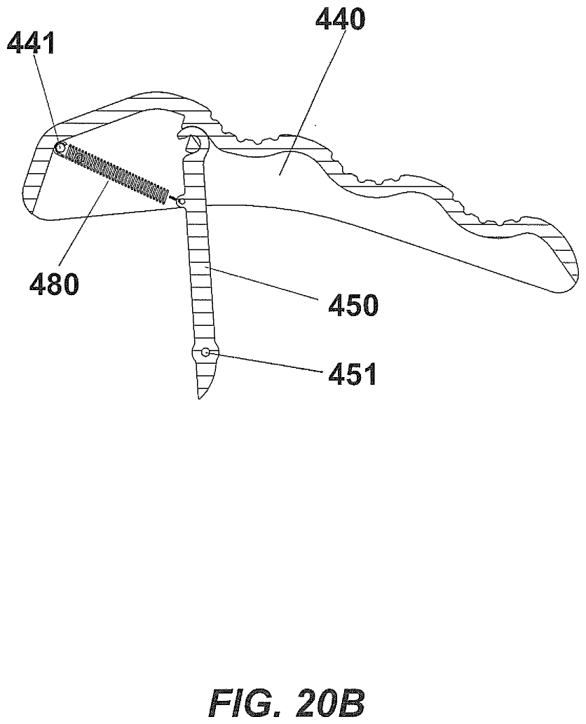

In other possible embodiments as shown in FIGS. 20B-C the housing 110 includes within it a support retraction rod or core shaft 169, which is connected to either the supports 160 themselves on the proximal end while the distal end of the supports 160 are connected to the mesh implant 300, and also a push or deployment rod 190 or other components of the inventive mesh implant device 100. In this embodiment there is a knob 165 at the proximal end of the support retraction rod 169 for use by the surgeon and there is also a stopper, gasket, plug, former or the like 167 located adjacent to the folded mesh implant 300, the plug 167 including openings or apertures (not shown) which correspond with the distal ends of the supports 160 when the mesh implant 300 is in the folded unactuated state. The plug 167 also functions as a safety by preventing the supports 160 from moving too far in any direction. Another embodiment not shown includes a plate, in this embodiment comprised of steel but could be comprised of any biocompatible material such as polymers or other metals or cap could be attached to the mesh implant 300, possibly with an adhesive or other connecting means, capable of pulling away from the mesh implant on force or retraction of the deployment rod 190 and the supports 160 form the mesh implant 300 once the mesh implant 300 is located on the target tissue area and released from the mesh implant device 100. The deployment rod 190 also has a plug 193 which functions as a safety feature and connects the deployment rod 190 with the support retraction rod 169. The support retraction rod 169 and deployment rod 190 also assist in centering the inventive mesh implant device 100 when inserted into the plug 193 prevents loss of pressure within the body cavity during the surgical procedure in that if any air or carbon dioxide gas that is placed into the abdominal cavity to create a working space escaped through the inventive mesh implant device 100 by leaking out within the housing to outside the body cavity then the surgical procedures itself is compromised and the abdomen will no longer be inflated with the carbon dioxide or pressured air. Loss of insufflation of the abdominal cavity, or pneumoperitoneum, would immediately stop the surgical procedure and endanger the patient, thus the plug 193 prevent loss of pneumoperitoneum. When the deployment rod 190 is pulled back out of the housing 100 after deployment of the folder mesh implant 300 there is a slide area or aperture 191 through which the length of the rods 169, 190 can move either with the other or without the other. Other functions of the plugs 193, 167 and the apertures 191 may be employed.

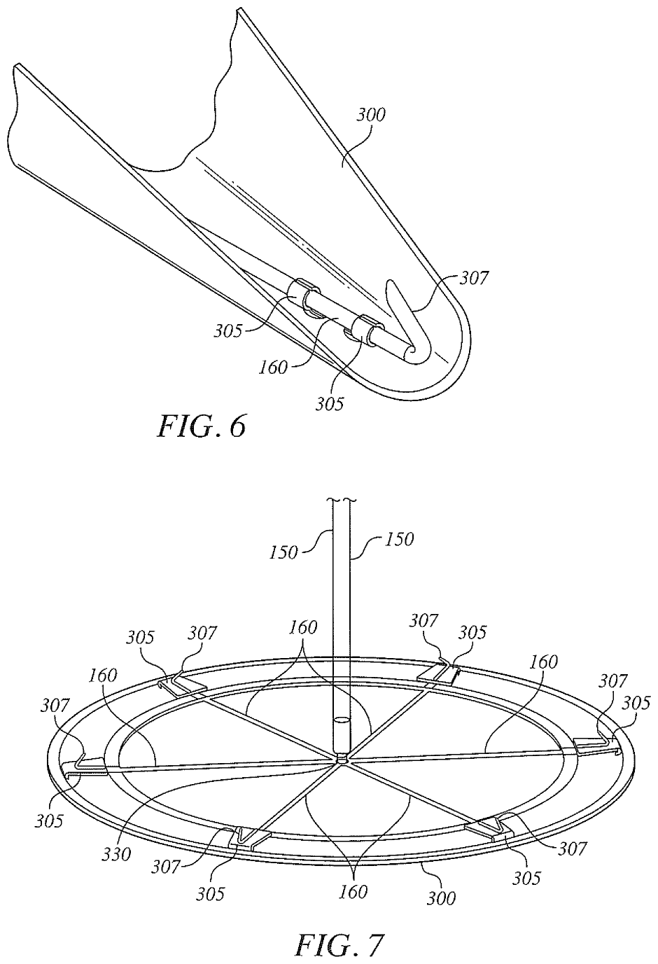

In one embodiment of the present invention the distal end of the supports 160 are configured in a hook shape so as to fasten the mesh implant 300 to the target tissue, as shown in FIGS. 6 and 7, and thus remain permanently connected to the mesh implant 300 and remain within the patient. In another embodiment of the present invention, the inventive fasteners connect the support 160 temporarily to the mesh implant 300 but upon sufficient pulling force the supports 160 are disconnected from the mesh implant 300 and retracted out of the housing (if not already retracted and removed itself) and the body cavity. The inventive fasteners 310 in such an embodiment remain and are used to fasten the mesh implant 300 to the target tissue. The other connecting means to fasten the mesh implant 300 to the target tissue may also be employed such as staples tacks, sutures, glue, and other conventional fasteners or fasteners as later developed in the art.

In yet another embodiment of the present invention the mesh implant 300 includes removable supports 160 which are not fastened to the outer edge of the mesh implant 300 but are only connected by looping through the suture 150 within the center of the mesh implant 300. In yet a further embodiment the distal end of the supports 160 may be temporarily fastened to the outer edge of the mesh implant 300 such that upon deployment and opening of the folded mesh implant 300 the support 160 assist to support the shape of the mesh implant 300 but such fastening means is detached from the mesh implant 300 with minimum force exerted so that the mesh implant 300 and suture 150 remain without any fasteners, such as shown in FIGS. 19-24. The mesh implant 300 in such an embodiment may include a plurality of apertures (not shown) along the outer edge of the mesh implant for insertion of separate fasteners to attach and implant the mesh implant 300 to the target tissue. In such an embodiment the fasteners may include without limitation tacks, staples, screws, nuts, bolts, sutures, glue and the like or combinations thereof. In yet another embodiment not shown, the mesh implant 300 may include tacks or other connecting means to connect the mesh implant 300 when deployed within the body cavity of the patient to attach to the target tissue.

The inventive mesh implant device 100 may further include a connecting means which may connect to the supports 160 at the point where such supports 160 pass over the center of the mesh implant 300. The connecting means 340 may function to tie the plurality of supports 160 together at one point so each support is not moving apart from the other prior to bending in a configuration along the surface of the folded or deployed mesh implant 300. It is contemplated that the connecting means could be a stainless steel plate, polymer or metal cap which could be adhered to the inner portion of the mesh implant 300. The connecting means may be comprised of any material compatible to the human body as it will be placed within the body cavity for deployment of the mesh implant 300. Such materials for the connecting means include plastics or metals and if a metal may be comprised of stainless steel and may further be coated with a shrink wrap plastic such as shrinkable polyethylene fiberglass, or polyvinyl chloride of a grade suitable for use in surgical procedures. The connecting means may be comprised of many known polymers such as polycarbonates or ABS. In one embodiment the connecting means is a plastic or metal rivet, crimp, open tie such as a circle, or washer or the like or combinations thereof. In another embodiment the connecting means is permanently connected to the mash implant 300 such as a rivet 330 as shown in FIGS. 4-5 and 7.

The inventive mesh implant device 100 further optionally includes a pre-loaded suture 150 to close the incision point above the fastened mesh implant 300, though the suture may not be included in certain embodiment such as when the inventive mesh implant device 100 is not inserted in the umbilicus or just above the target tissue location, but rather such as by way of non-limiting example in robotic surgery, the inventive mesh implant device 100 is inserted within the abdomen at a location below the target tissue of hernia and therefore the mesh implant 300 would not include the suture 150. In the alternative, even if the suture 150 is included in the mesh implant 300, but the inventive mesh implant device 100 is not in itself being used to close the port opening incision, the suture 150 could be cut and pulled out of the mesh implant 300. The suture 150 may be comprised of silk, vicryl, davdelk, polypropylene, polymers or any other conventional thread or tying material. The suture 150 is loaded into the housing 110 so that it starts on one side, is housed within the hollow channel of the housing 110, is connected to the center of the mesh implant 300 typically through two apertures located near the center of the mesh implant 300 though other locations on the mesh implant 300 and more than two apertures may be employed, and the other end is housed on the other side of the housing 110 as shown in FIGS. 1, 3, and 8-15. The suture 150 extends out of the proximal end of the housing 110 so that upon deployment and fastening of the mesh implant 300 to the target tissue and retraction and removal of the supports 160 the surgeon can suture the port or incision point closed with the pre-loaded suture 150.

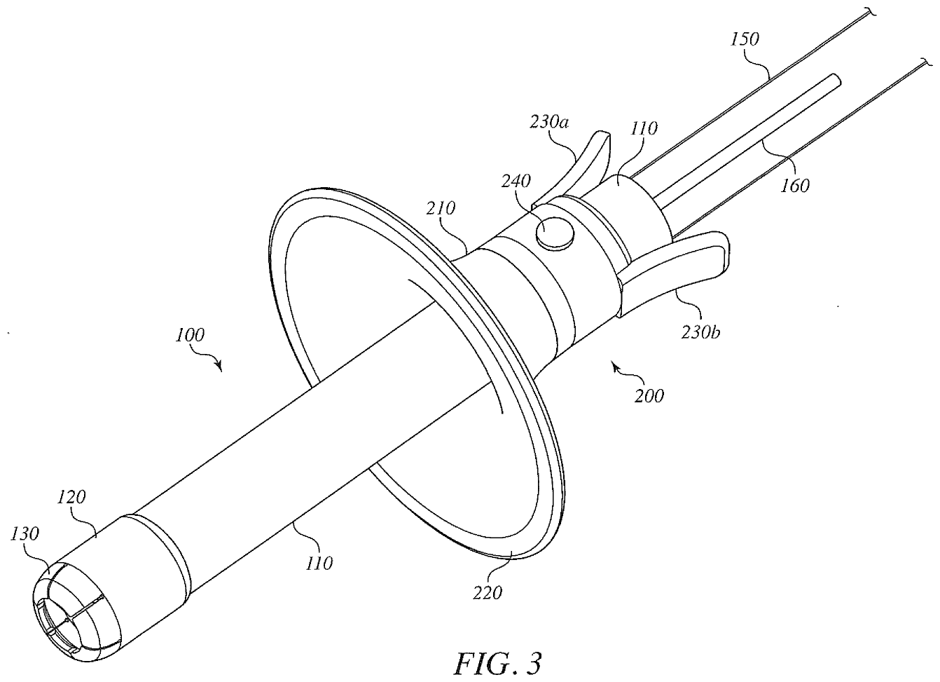

The inventive mesh implant device 100 may optionally include a stabilization device 200 as shown in FIGS. 3, 9 and 11-12. The stabilization device 200 has a housing 210 which is vertically movable on the outer surface of the housing 110 during surgery. The stabilization device 200 may be lowered vertically so as to rest on the outer fascia of the patient at the incision point or port. The stabilization device 200 may be comprised of any material, preferably compatible to the human body though it will not reside within the body cavity but rest on the outer fascia. Preferred materials are plastics or metals. The stabilization device 200 has a wider bottom surface 220 for stabilization purposes and has in one embodiment two grips 230a, 230b to facilitate the downward and upward movement on the housing 110. The grips 230a, 230b may be comprised of plastics or metals. The grips 230a, 230b are connected to the stabilization device 200 via a connecting means 240 such as a pin, screw, bolt, nut, crimp, solder, or any connecting means. If the stabilization device 200 is a metal it may be comprised of stainless steel and may further be coated with a shrink wrap plastic such as shrinkable polyethylene fiberglass, or polyvinyl chloride of a grade suitable for use in surgical procedures. The stabilization device 200 may be comprised of many known polymers such as polycarbonates or ABS. For cost effectiveness, the stabilization device 200 may be comprised of any plastic capable of sterilization according to regulatory agencies for medical devices.

In one embodiment of the present invention the mesh implant device 100 includes a handle 400 on the housing 110, as shown in FIGS. 19-24. The handle 400 may be comprised of any material, preferably compatible to the human body though it will not reside within the body cavity but is on the housing 110. Preferred materials are plastics or metals. The handle 400 assists the surgeon in positioning the mesh implant device 100 and facilitate moving the push rod 190 in an upward and downward motion to activate and deploy the mesh implant 300 as well as activate and retract the supports 160 after the mesh implant 300 is positioned at the target tissue. In this embodiment the handle 400 has a handle housing 420 and a lever 440. The lever 440 is also used to hold the proximal end of the optional suture 150 (not shown) which can be pulled taut after the mesh implant 300 is deployed. The lever 440 is also connected to a proximal end of the supports 160 so that when activated (in a downward direction of the lever 440), the supports 160 are retracted and withdrawn back into the housing 110 and out of the body cavity. Other versions of a handle may be employed to make the grip easier for the surgeon or medical profession when in use.

In one embodiment of the present inventive mesh implant device 100, the lever 440 is injection molded plastic and includes an internal triangle 460 (though any other shape besides a triangle may be employed) that is molded by two side actions in the mold. The molding processes allows the hook end of a stamped or laser cut stainless steel pawl 450 to be quickly rotated in place during its assembly operation thereby eliminating the need for an inserted pin through the lever 440 and the associated potential device failure of a pin falling out during use. The design fool proofs the orientation of the pawl 450 as a backwards pawl 450 is not able to fit in the lever 440. Further, the inventive design of the handle 400 with lever 440 also improves the entire inventive mesh implant device 100 esthetics with no externally visible pin. Assembly costs are also significantly reduced with the elimination of a pin and the design is more suitable for automated high volume assembly. While in this embodiment a pawl 450 is employed, any other hinged catch that fits into a notch of a ratchet to move a wheel forward or prevent it from moving backward can be used or any other ratchet mechanism in general. As shows in in FIGS. 20A-B, the lever 440 is attached to the handle 400 via a pin 441 so that the lever 440 can be pushed in a downward direction towards the handle 400. The shape and length of the pawl 450 affects the downward ratcheting as well as the shape of the tip of the pawl 451. The ratchet and wheel are not shown but the lever may be cycled a number of times so as to retract the support 160 from the mesh implant 300 and back into the housing 110 of the inventive mesh implant device 100.

In the embodiment as shown in FIGS. 19-24 and more specifically in FIGS. 22C-D, the mesh implant 300 includes a small aperture 322 through which a cap 320 is inserted so that the diameter of the cap 320 is slightly larger than the aperture 322 but when the supports 160 connected to the cap 320 are retracted and withdrawn the cap 320 separates from the mesh implant 300 through force without tearing of the mesh implant 300. If the optional suture 150 is included on the mesh implant 300, the suture 150 is connected (or referred to as threaded) on a location outside of the outer circumference of the aperture 322. In other embodiments it is contemplated that the mesh implant 300 is connected to a connecting means with an adhesive so that the connecting means helps to shape the mesh implant 300 within the housing in the inactivated and stored position and to provide shape for the distal end of the supports 160. The connecting means in those embodiments is easily removed or disassociated from the mesh implant 300 for force when the supports 160 are being retracted from the mesh implant 300.

In another embodiment not shown the mesh implant 300 may have a skirt imprinted on the outer edge for visualization by the surgeon of the area in which to secure the deployed mesh implant 300 to the target tissue, such as for example via tacks (not shown). In use, the mesh implant 300 once deployed and set along the target tissue with the supports 160 retracted and removed from the body cavity, is then tacked or sutured to the target tissue via a separate device inserted into the body cavity at a different incision point into the abdominal cavity. Other connecting means such as sutures, staples and the like may also be employed to connect the opened mesh implant 300 to the target tissue.

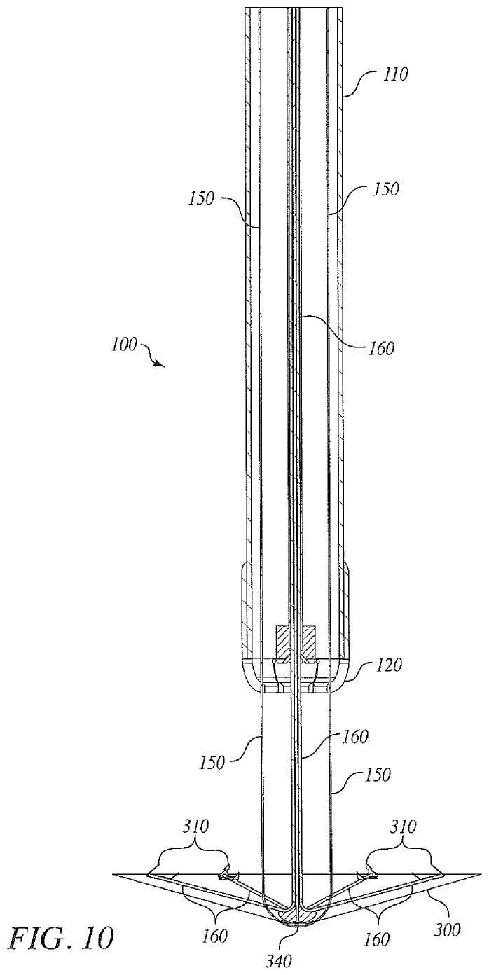

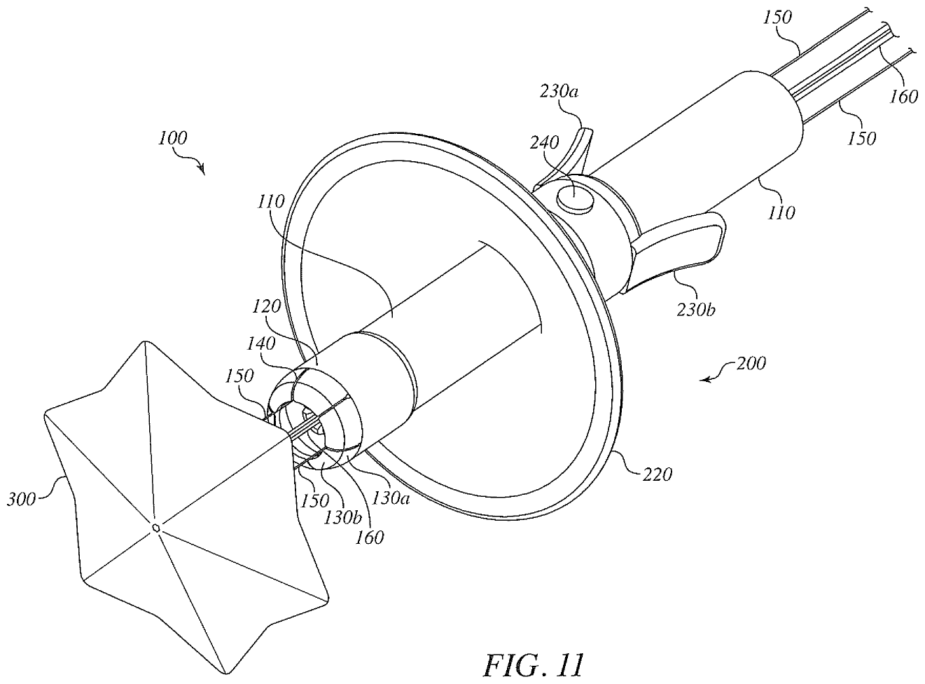

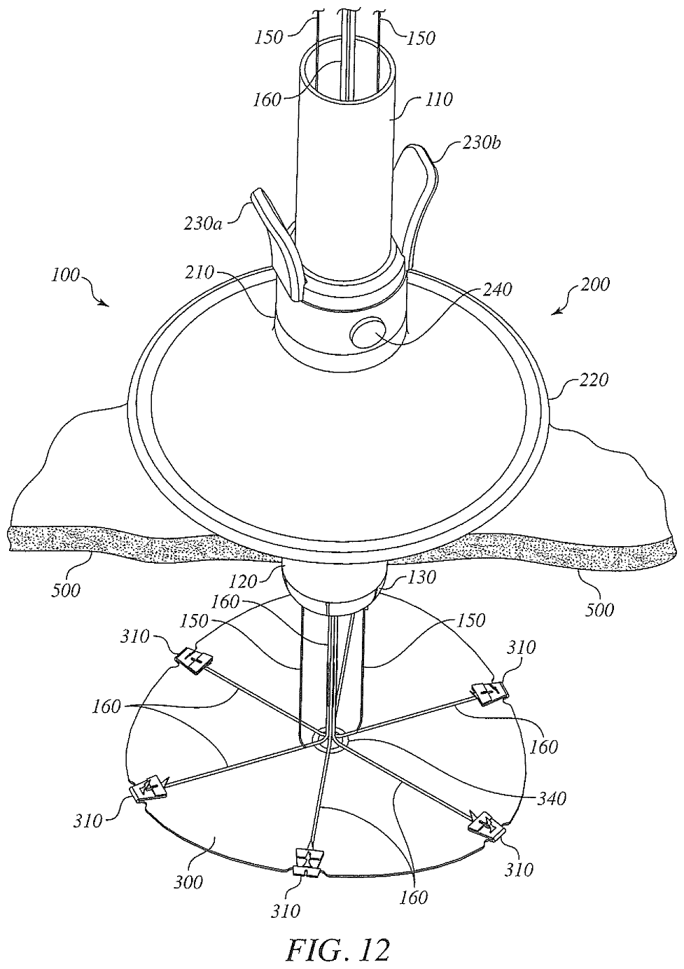

A method of use of the inventive mesh implant device 100 includes the steps of inserting the mesh implant device 100 through an incision port or point in the fascia 500 of a patient, such as the umbilicus 510 as shown in FIGS. 12-16. Optionally no other surgical instrument or trocar is within the incision point or port upon insertion of the inventive mesh implant device 100, though in one embodiment of the inventive method the mesh implant device 100 may be inserted through a trocar with the trocar later removed prior to suturing of the incision port. Further, the cap 320 on the most distal end of the inventive mesh implant device 100 may aid in inserting the inventive mesh implant device 100 within the fascia 500 as well as the umbilicus 510 as it is either atraumatic or traumatic. Next the distal end of the housing 110 (in FIGS. 1-17 includes the distal end 130 and cap 120) are placed below or near the target location of the tissue to which the non-deployed and unactuated mesh implant 300, which is housed and folded within the housing 110, will be fastened and implanted. If the surgeon is using the inventive mesh implant device 100 to close an incision port then the distal end of the housing 110 (and cap 120 if applicable) of the housing 110 are inserted within the incision port or point in the fascia 500 a sufficient amount such that the mesh implant 300 may be deployed safely within the body or body cavity to be fastened to the tissue on each side of the incision port or point. The deployment mechanism, such as a push rod 190 is activated by pushing it towards the distal end of the inventive mesh implant device 100 and the folded unactuated mesh implant 300 is deployed from within the interior of the housing 110 and if applicable through the cap 120 if applicable and optionally the distal end 130 and indentations 140 of the cap 120. This movement also pushes or extends the distal end of the supports 160 and the suture 150, both attached to the mesh implant 300 (at the center of the mesh implant 300 or one or more other locations on the mesh implant 300), out of the distal end of the housing 100. The mesh implant 300 in its partially deployed state or position resembles the shape of an umbrella in one embodiment of the invention as shown in FIGS. 10 through 13. The housing 110 may be retracted out of the body and fascia 500 at this time in the method, or may be retracted or removed upon attachment or fastening of the mesh implant 300 to the target tissue. As shown in FIG. 14 the housing 110 has been removed from the fascia 500 and the deployed mesh implant 300 and the supports 160 and the suture 150 remain within the body. In one embodiment of the invention the distal ends of the supports 160 may include a hook 307 or other connecting means and are used to connect or fasten the mesh implant 300 to the target tissue.

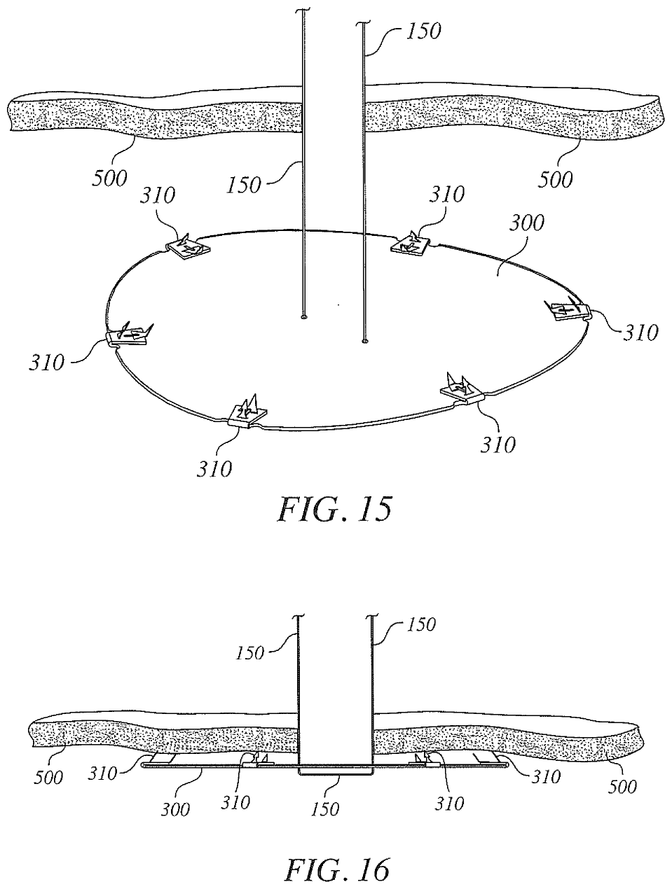

The mesh implant 300 in its fully deployed state or position resembles the shape of a circle in one embodiment of the invention as shown in FIG. 14. In another embodiment of the present invention wherein the supports 160 do not include fasteners (305 or 310) but rather the mesh implant 300 includes separate fasteners (not shown) the mesh implant 300 is then moved into a position for attachment or fastening to the target tissue, typically in an upward or side direction depending on the location of the incision port or point on the fascia 500. The supports 160 are then retracted and removed, such as by force or other mechanisms. As shown In FIG. 15 the deployed mesh implant 300 and the fasteners 310 and the suture 150 remain in the body. As shown in FIG. 16, the mesh implant 300 is then attached and fastened to the target tissue, whether it is a hernia or the sides of an incision port or other target location, via the fasteners 310.

In each embodiment of the present invention including the optional suture 150, once the mesh implant 300 is sufficiently fastened to the target tissue the suture is then employed by the surgeon to dose the incision port. The ends of the single suture 150 are outside of the fascia 500 with a portion of the suture 150 connected to, in one embodiment of the inventive port closure device 100, the center of the mesh implant 300. The surgeon then pulls the ends of the suture 150 with force and knots or otherwise closes the suture and thus closes the incision point or port. Thus no additional instruments may be needed to close the incision port or point. Additional sutures may be employed by the surgeon to close the incision point or port though not necessary.

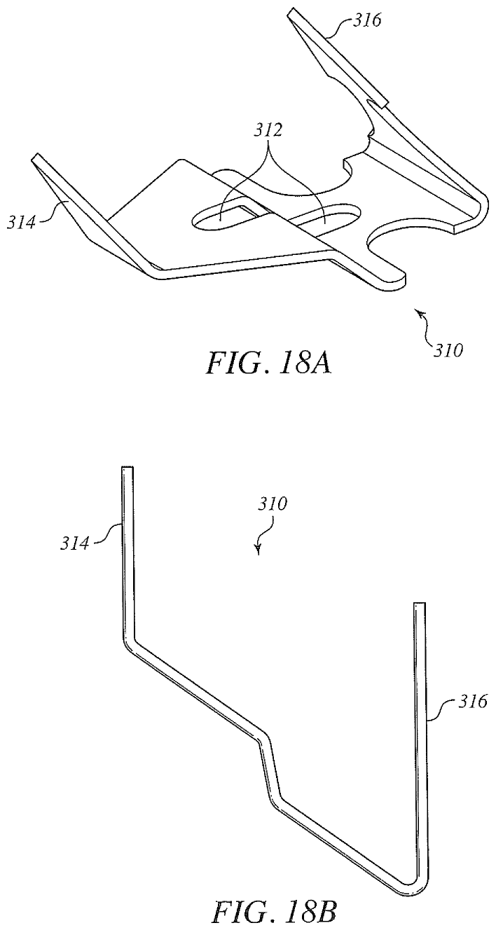

A further inventive component of the present invention is an inventive fastener 310, shown in FIGS. 18A and 18B, which is configured so that a distal end of the support 160 is retained within an aperture 312 of a proximal end 314 of the fastener 310 but said distal end of the support 160 may be detached and released from the aperture 312 upon sufficient force such that the support 160 may be removed from the mesh implant and the fascia 500 through the incision port, or optionally through the housing 110 if not previously removed. The distal end 316 of the fastener 310 extends from the mesh implant 300 and is configured to connect the mesh implant 300 to the target tissue. Many shapes and forms of the inventive fastener are possible including without limitation the one depicted in the figures.

Yet another method of the present invention is shown in FIGS. 23A-G using the inventive mesh implant device 100 of FIG. 19. The inventive mesh implant device 100 is inserted into the fascia 500 in this example via the umbilicus 510 (FIG. 23A). The mesh implant 300 is deployed by pushing on the deployment rod 190, possibly via the knob 195 or the surgeon can just move the entire deployment rod 190 which at this time is connected to the support retraction rod 169 (FIG. 23B). Note the two knobs 165, 195 are joined in this embodiment until such time as the supports 160 are to be retracted form the mesh implant 300 and then the knob 165 on the proximal end of the support retraction rod 169 is turned 30-90 degrees and they are separated such that the support retraction rod 169 can be pulled away from the deployment rod 190. FIG. 23B shows the folded mesh implant 300 just deployed out of the housing 110 and the supports 160 are just beginning to spring open, which may last less than a second. In FIG. 23C the mesh implant 300 is fully deployed and in the open actuated state or position and in this embodiment is at a right angle from the deployment rod 190 and the housing 110. The lever 440 in FIGS. 23A-E is still in the upward position as in FIG. 23D the housing 110 and entire inventive mesh implant device 100 is pulled out partially from the body cavity to move the deployed mesh implant 300 closer to the target tissue as shown in FIG. 23E. The support retraction rod 169 is then activated by turning the knob 195 in this non-limiting example 30 degrees and uncouples from the deployment rod knob 195. The lever 440 is then cycled or pumped in a downward direction towards the housing 110 and handle housing 420 a set number of times dependent on the wheel and ratcheting measurement (not shown) via the pawl 450, and in this embodiment it is seven times thereby retracting the supports 160 from the mesh implant 300. The retraction of the supports 160 will not occur until such time as the mesh implant 300 is secured over the target tissue or hernia 550 via sutures, tacks, and other known fasteners. Finally the entire inventive mesh implant device is removed from the body cavity and fascia 500 as shown in FIG. 23G. If the mesh implant 300 has an optional suture 150 as shown in FIGS. 1-16 the umbilicus 510 is closed with the suture 150.

Another aspect of the present invention is a surgical kit which includes the inventive mesh implant device 100. The kit is stored in a sterile sealed package. The kit may include a trocar, a scissors device, a grasper device, a specimen retrieval device and the mesh implant device 100. The kit may optionally include a cauterizing device such as a bi-polar device. Other optional devices may be included. In one embodiment of the inventive kit all components are single use only and disposable. In another embodiment of the inventive kit some components are single use and disposable while others are reusable (typically after sterilization). In yet another embodiment of the inventive kit the components are reusable (typically after sterilization) except that the bag would be single use only and disposable.

Advantages of the inventive mesh implant device 100 include the configuration such that it is easier to close an incision port or point on the fascia of a patient with the optional suture 150. Accordingly, the incision location is smaller and may cause less surgical damage to the fascia, reduce the total operation time required for the procedure and reduce possible complications. Notably, in any of the embodiments, as only one surgical instrument and/or device is needed to insert the mesh implant 300 the surgical process for repairing a defect in the body cavity such as a hernia can be simplified by using the inventive mesh implant device 100 and the time and cost for the surgery can be reduced. Typically, the greatest amount of time during a hernia surgery is unwinding the mesh implant within the body cavity, moving it with graspers or other surgical instruments into the target are and holding the mesh implant taut at the target area which then suturing the mesh to the inner fascia 500 of the patient, which can take as long as 1-2 hours for just this step alone in the overall surgery. By deploying the inventive mesh implant device 100, the surgical time can be significantly shortened resulting in less complications for the patient as well as cost savings for quicker surgical time.

Further, the configuration of the inventive mesh implant device 100 enables it to, compared to conventional surgical instruments and devices, better stabilize the area of surgery, deploy or activate the mesh implant, connect the deployed mesh implant to the tissue via conventional or inventive fasteners, and if using the optional suture 150 close the incision point or port, all with one device. The inventive mesh implant device may be used for a variety of surgical procedures, such as a hernia, but may also be used solely to close a surgical incision port or point, such as at the umbilicus or any incision point on a patient's fascia or within a body cavity or elsewhere. The inventive device and inventive methods may decrease surgical time, decrease the number of surgical instrument within the incision point, decrease the potential complications and decrease pain for the patient. Overall, the mesh implant device 100 may reduce complications, surgical processes, time and cost.

Many possible combinations could be within the mesh implant device, the methods of use, the method of treatment and the kit or system of the present invention. For example, the kit could include bandages and other dressings to apply to the incision location post-surgery. The kit could include elevation apparatus or devices such as a pre-filled air bladder as disclosed in co-owned pending applications and issued patents such as PCT/US23/25642 or compression apparatus and devices as disclosed in co-owned pending applications and issued patents. Other dressings, apparatus and devices would be included in the kit.

Another embodiment of the inventive method using the inventive mesh implant device 100 appears to be more efficient is taking a pre-assembled mesh device that comes in various shapes and sizes, and inserting it into the abdominal cavity. It would then be deployed like an umbrella. The opened mesh “umbrella” would then be placed up against the abdominal wall, and the edges tacked with existing tackers. The supports 160 or spines would be retracted, leaving the mesh implant 300 attached to the abdominal wall. The method steps in this embodiment include: (1) The exhibited device is approximately 27 mm in diameter and it carries a 20×15 cm mesh implant 300. The device 100 size can be reduced to a 15 mm diameter for smaller mesh implant 300 sizes. (2) The device has a pre-loaded folded mesh implant 300 inside the housing 110 or tube including six Nitinol wires (supports 160) that are threaded tangentially into the mesh implant 300. Nitinol wire is able to retain any desired shape. (3) The device 100 is inserted into the abdominal cavity under direct vision and once confirmed, the deployment rod 190 is advanced forward to deploy the mesh implant 300 from inside the housing 110 or tube. The mesh implant 300 is now opened as an umbrella typically at a right angle of about 90 degrees compared to the housing 110, with nitinol wire retaining the shape and stiffness. (4) With the mesh implant 300 completely open the entire device 100 is pulled back until the mesh implant 300 abuts the peritoneum inside the abdominal wall. (5) The suture 150 coming from the center of the mesh implant 300 is pulled back and kept taut. (6) Surgical tacks are placed into the mesh implant 300 against the abdominal wall 500, until the mesh implant 300 is fully secured. (7) The deployment rod 190 or plunger is rotated counter-clockwise to lock it against the housing 110 or tube, which frees the supports 160 or spines for withdrawal. (8) The supports 160 or spines are withdrawn horizontally out of the mesh implant 300 into the housing 110 or tube by squeezing the handle 400 about 7-8 times. (9) This leaves the tacked mesh implant 300 closing the hernia defect circumferentially. (10) The whole device 100 is then withdrawn from the abdominal cavity with proper tension on the suture 150. (11) Finally, the suture 150 from the mesh implant 300 is sewed to the fascia 500 where the device 100 was inserted. This helps to close the fascia 500 over the mesh implant 300 and anchors the mesh implant 300 to the middle of the defect preventing any tenting/migration. The skin 500 is closed at this point with the mesh implant 300 secured to the target tissue within the body cavity.

In another embodiment of the inventive method using the inventive mesh implant device 100 as shown in FIGS. 25A-25C, the inventive mesh implant device 100 includes a pre-assembled mesh device that comes in various shapes and sizes with the housing 110 of the device 100 capable of being inserted into the abdominal cavity when the mesh implant 300 would then be deployed like an umbrella. The opened mesh “umbrella” would then be placed up against the abdominal wall, and the edges tacked with existing tackers. Surgical tacks (not shown) are placed into the mesh implant 300 against the abdominal wall, until the mesh implant 300 is fully secured. The knobs 165, 195, are rotated either together or against each other such as counter-clockwise to lock it the deployment rod 190 against the housing 110, which frees the supports 160 or spines for withdrawal. The supports 160 or spines are withdrawn horizontally out of the mesh implant 300 into the housing 110 or tube by squeezing the lever 440 or further rotating one or both of the knobs 165, 195 a number of times such as 2-20. After the mesh implant 300 is secured to the target tissue or hernia 550, the whole device 100 is then withdrawn from the abdominal cavity, applying proper tension on the optional suture 150 if employed and then the optional suture 150 from the mesh implant 300 is sewed to the fascia 500 where the device 100 was inserted thus closing the port or incision. This helps to close the fascia 500 over the mesh implant 300 and anchors the mesh implant 300 to the middle of the defect preventing any tenting/migration. The ski or fascia 500 is closed at this point with the mesh implant 300 secured to the target tissue within the body cavity and need not only be at the umbilicus 510.

In yet another embodiment of the inventive method using the inventive mesh implant device 100 as shown in FIG. 24, the inventive mesh implant device 100 includes a mesh implant 300 that when deployed like an umbrella but only in a partially open position or state. The opened mesh “umbrella” would then be moved to the location over the target tissue or hernia within the body cavity, fully opened into a right angle about 90 degrees compared to the housing 110 or even at a higher degree such as an inverted umbrella, then placed up against the abdominal wall, and the edges of the mesh implant 300 tacked with sutures, staples, tacks or other connecting means. The supports 160 or spines would be retracted, leaving the mesh implant 300 attached to the abdominal wall and in this embodiment the mesh implant 300 does not include a suture 150 to close the port so that the incision port is closed separately from the withdrawn inventive mesh implant device 100.

In another embodiment of the present invention using another embodiment of the inventive device for example for use in robotic surgery. The inventive fastener further provides an improved manner to fasten a mesh implant to a target tissue or location within or outside of a body cavity or other location for a patient (human or animal) in an easier way.

Although the exemplary embodiments of the present invention have been described, it is understood that the present invention should not be limited to these exemplary embodiments but various changes and modifications can be made by one ordinary skilled in the art within the spirit and scope of the present invention as hereinafter claimed. The invention has been described in terms of embodiments thereof, but is more broadly applicable as will be understood by those skilled in the art.

I claim:

1. A mesh implant device for use in laparoscopic procedures; comprising:
a hollow housing;
a cone assembly located at a distal end of the hollow housing
a mesh implant having a foldable mesh material connected via an aperture to a cap on the top of the cone assembly;
a plurality of supports connected but detachable to the mesh implant;
the cap to which is held the plurality of supports, the hollow cap being at a distal end of the hollow housing and having at least an opposite pair of grooves;
wherein the mesh implant and said housing are configured and arranged so that the mesh implant moves between a stowed condition within the housing and a deployed condition outside the housing, the plurality of supports being configured and arranged to move between a retracted position that folds the mesh material to enable placement of the mesh implant in the stowed condition and an extended position outside the housing that unfolds the mesh material to enable placement of the mesh implant in the deployed condition.

2. The mesh implant device of claim 1, further comprising a stabilization device movable along an exterior of the hollow housing between multiple relative positions, the stabilization means has grips that are movable between a grasping condition that grasps the hollow housing is a secure manner to prevent relative movement of the stabilization device on the hollow housing and a released condition that releases the grips from the grasping condition to permit relative movement of the stabilization device on the hollow housing.

3. The mesh implant device of claim 1, wherein the cone assembly includes ______.

4. The mesh implant device of claim 1, further comprising a deployment mechanism in operative connection with the mesh implant to deploy the mesh implant.

5. The mesh implant device claim 4, wherein the deployment mechanism is selected from the group consisting of a push rod, pull rod, thumb grip and a pistol grip deployment mechanism.

6. A surgical instrument and assembly for laparoscopic procedures; comprising:
a port closure device having a hollow housing;
a mesh implant having a foldable mesh material;
a plurality of supports;
a hollow cap to which is held the plurality of supports, the hollow cap being at a distal end of the hollow housing and having at least an opposite pair of grooves;
a plurality of fasteners attached to the plurality of supports in a manner that fastens the plurality of supports to the mesh material; and
at least one suture thread that extends through the hollow housing to the cap;
wherein the mesh implant and said housing are configured and arranged so that the mesh implant moves between a stowed condition within the housing and a deployed condition outside the housing, the plurality of supports being configured and arranged to move between a retracted position that folds the mesh material to enable placement of the mesh implant in the stowed condition and an extended position outside the housing that unfolds the mesh material to enable placement of the mesh implant in the deployed condition, the suture thread extending across the opposite pairs of grooves with the mesh implant in the stowed condition and extending across a center of the mesh with the mesh implant in the deployed position.

7. The surgical instrument and assembly of claim 6, further comprising a stabilization device movable along an exterior of the hollow housing between multiple relative positions, the stabilization means has grips that are movable between a grasping condition that grasps the hollow housing is a secure manner to prevent relative movement of the stabilization device on the hollow housing and a released condition that releases the grips from the grasping condition to permit relative movement of the stabilization device on the hollow housing.

8. The surgical instrument and assembly of claim 6, wherein the stabilization device has opposite ends with one of the opposite ends being wider than a remaining one of the opposite ends, a wider one of the opposite ends being arranged closer to the cap than is the remaining one of the opposite ends.

9. The surgical instrument and assembly of claim 6, wherein the fasteners are hooks, the mesh material having crimps, the supports connecting to the mesh material by engagement of the hooks and crimps with each other.

10. The surgical instrument and assembly of claim 6, wherein the fasteners have one horizontal surface attached to the mesh implant and a connecting means extending from the opposite side of the fastener at an angle of between about 10 degrees to about 90 degrees compared to the horizontal surface with the connecting means including an aperture, the supports connecting to the mesh material by engagement of the aperture within the fasteners.

11. The surgical instrument and assembly of claim 6, further comprising a deployment mechanism in operative connection with the mesh implant to deploy the mesh implant.

12. The surgical instrument and assembly of claim 11, wherein the deployment mechanism is selected from the group consisting of a push rod, pull rod, thumb grip and a pistol grip deployment mechanism.

13. A method of assembling a surgical instrument for laparoscopic procedures comprising:
providing components of a surgical instrument, the components including (a) a mesh implant device having a hollow housing, (b) a mesh implant having a foldable mesh material attached to a deployment mechanism, (c) a plurality of supports, and (d) a hollow cap to which is held the plurality of supports, the hollow cap being at a distal end of the hollow housing and having at least an opposite pair of grooves;
configuring and arranging the mesh implant and said housing so that the mesh implant moves between a stowed condition within the housing and a deployed condition outside the housing;
configuring and arranging the plurality of supports to move between a retracted position that folds the mesh material to enable placement of the mesh implant in the stowed condition and an extended position outside the housing that unfolds the mesh material to enable placement of the mesh implant in the deployed condition.

14. The method of claim 13, further comprising; moving a stabilization device along an exterior of the hollow housing between multiple relative positions; and moving grips of the stabilization means between a grasping condition that grasps the hollow housing is a secure manner to prevent relative movement of the stabilization device on the hollow housing and a released condition that releases the grips from the grasping condition to permit relative movement of the stabilization device on the hollow housing.

15. The method of claim 14, wherein the stabilization device has opposite ends with one of the opposite ends being wider than a remaining one of the opposite ends, further comprising: arranging a wider one of the opposite ends of the stabilization device closer to the cap than is the remaining one of the opposite ends.

16. The method of claim 13, further comprising attaching the mesh implant to the target body area via fasteners selected from the group consisting of hooks, crimps, tacks, sutures, adhesive and combinations thereof with each other.

17. The method of claim 16, wherein the fasteners have one horizontal surface attached to the mesh implant and a connecting means extending from the opposite side of the fastener at an angle of between about 10 degrees to about 90 degrees compared to the horizontal surface with the connecting means including an aperture, further comprising: attaching the supports to the mesh material by engagement of the aperture within the fasteners.

18. The method of claim 13, further comprising operating a deployment mechanism in operative connection with the mesh implant to deploy the mesh implant.

19. The method of claim 18, wherein the deployment mechanism is selected from the group consisting of a push rod, pull rod. thumb grip and a pistol grip deployment mechanism.
